(19) United States
(12) Patent Application Publication
(10) Pub. No.: US 2017/0267975 A1
(43) Pub. Date: Sep. 21, 2017

(54) THREE-DIMENSIONAL CULTURE STRUCTURE AND METHOD FOR PRODUCING SAME

(71) Applicants: Aino Hasegawa, Kanagawa (JP); Daisuke Takagi, Kanagawa (JP); Shigeo Hatada, Kanagawa (JP); Yuuma Usui, Kanagawa (JP); Chihiro Kubo, Kanagawa (JP); Shusaku Shiomoto, Kanagawa (JP); Waka Lin, Tokyo (JP)

(72) Inventors: Aino Hasegawa, Kanagawa (JP); Daisuke Takagi, Kanagawa (JP); Shigeo Hatada, Kanagawa (JP); Yuuma Usui, Kanagawa (JP); Chihiro Kubo, Kanagawa (JP); Shusaku Shiomoto, Kanagawa (JP); Waka Lin, Tokyo (JP)

(73) Assignee: RICOH COMPANY, LTD., Tokyo (JP)

(21) Appl. No.: 15/459,146

(22) Filed: Mar. 15, 2017

(30) Foreign Application Priority Data
Mar. 17, 2016 (JP) 2016-054442
Mar. 2, 2017 (JP) 2017-039615

(51) Int. Cl. C12N 5/077

(52) U.S. Cl. CPC C12N 5/0656; C12N 2513/00; C12N 2533/70; C12N 2533/90; C12N 2533/20; C12N 2533/72; C12N 2533/74

(57) ABSTRACT

Provided is a three-dimensional culture structure that is excellent in cell adhesion and cell stretching and can be produced efficiently. The three-dimensional culture structure includes cells, a cell support material configured to support the cells, and bioaffinity particles. In a preferable mode, the bioaffinity particles are exposed from at least part of the surface of the cell support material, or the bioaffinity particles are protruded from the cell support material, or a surface area occupation rate at which the bioaffinity particles are exposed is 20% or greater of the entire surface of the three-dimensional culture structure, or the bioaffinity particles are dispersed in the cell support material.

### CROSS-REFERENCE TO RELATED APPLICATIONS

The present application claims priority under 35 U.S.C. §119 to Japanese Patent Application No. 2016-054442, filed Mar. 17, 2016 and Japanese Patent Application No. 2017-039615, filed Mar. 2, 2017. The contents of which are incorporated herein by reference in their entirety.

### BACKGROUND OF THE INVENTION

Field of the Invention

The present invention relates to a three-dimensional culture structure for cell culture and a method for producing the same.

Description of the Related Art

Recently, in the field of cell culture technology, a method for inducing regeneration of a damaged living tissue using a cell sheet that is produced in vitro by placing cells on a base material has been developed. The cell sheet is produced by coating a surface of a base material with a temperature-responsive polymer having a lower critical solution temperature, raising the temperature to higher than the lower critical solution temperature of the polymer to turn the sol-state polymer to a gel state, and culturing cells on the gel-state polymer. It is possible to peel the produced cell sheet from the polymer on the base material by lowering the temperature to lower than or equal to the lower critical solution temperature of the polymer to cause a phase change of the polymer from the gel state to the sol state.

However, there is a problem in the production of the cell sheet. Adhesion and stretching of the cells do not make progress on the polymer, and it is impossible to obtain the cell sheet efficiently at a high productivity.

Hence, there is proposed a cell aggregate containing cultured cells and gelatin hydrogel particles (see, e.g., International Publication No. WO 2011/059112).

There is also proposed a bone regeneration graft formed of a porous three-dimensional structure containing a biopolymer having bioaffinity and a biodegradable polymer having a mechanical strength (see, e.g., Japanese Patent No. 4412537).

### SUMMARY OF THE INVENTION

According to one aspect of the present invention, a three-dimensional culture structure includes cells, a cell support material configured to support the cells, and bioaffinity particles.

### BRIEF DESCRIPTION OF THE DRAWINGS

FIG. 1A is an atomic force microscope (AFM) photograph of bioaffinity particles exposed at a surface area occupation rate of 1%;

FIG. 1B is an atomic force microscope (AFM) photograph of bioaffinity particles exposed at a surface area occupation rate of 18%;

FIG. 1C is an atomic force microscope (AFM) photograph of bioaffinity particles exposed at a surface area occupation rate of from 50% through 58%;

FIG. 2 is a graph plotting viscosity of a liquid containing gelatin particles and viscosity of a liquid containing gelatin;

FIG. 3A is a schematic view illustrating an example of a producing apparatus configured to produce a three-dimensional culture structure used in the present invention;

FIG. 3B is a schematic view illustrating another example of a producing apparatus configured to produce a three-dimensional culture structure used in the present invention;

FIG. 4 is a graph plotting change in the particle size distribution of gelatin particles depending on change in the content of gelatin during gelatin particle production;

FIG. 5 is a scanning electron microscope (SEM) image of gelatin particles in a dry state;

FIG. 6 is a graph plotting change in the particle size distribution of gelatin particles depending on change in the content of a cross-linking agent during gelatin particle production;

FIG. 7A is a photograph presenting a state of cells after cell culture for 24 hours in Comparative Example 1;

FIG. 7B is a photograph presenting a state of cells after cell culture for 24 hours in Comparative Example 1;

FIG. 7C is a photograph presenting a state of cells after cell culture for 24 hours in Comparative Example 2;

FIG. 7D is a photograph presenting a state of cells after cell culture for 24 hours in Comparative Example 2;

FIG. 7E is a photograph presenting a state of cells after cell culture for 24 hours in Comparative Example 3;

FIG. 7F is a photograph presenting a state of cells after cell culture for 24 hours in Example 4;

FIG. 8A is a photograph presenting a state of cells after cell culture for 48 hours in Comparative Example 1;

FIG. 8B is a photograph presenting a state of cells after cell culture for 48 hours in Example 1;

FIG. 8C is a photograph presenting a state of cells after cell culture for 48 hours in Example 2;

FIG. 8D is a photograph presenting a state of cells after cell culture for 48 hours in Comparative Example 2;

FIG. 8E is a photograph presenting a state of cells after cell culture for 48 hours in Example 3;

FIG. 8F is a photograph presenting a state of cells after cell culture for 48 hours in Example 4;

FIG. 9A is a photograph presenting a state of cells after culture for 48 hours in Example 1;

FIG. 9B is a partially enlarged photograph presenting a solid line portion in FIG. 9A;

FIG. 9C is a partially enlarged photograph presenting a solid line portion in FIG. 9B;

FIG. 10A is a photograph presenting a state of cells after cell culture for 4 hours in Example 5;

FIG. 10B is a photograph presenting a state of cells after cell culture for 24 hours in Example 5;

FIG. 11A is a photograph presenting a state of cells after cell culture for 4 hours in Example 6;

FIG. 11B is a photograph presenting a state of cells after cell culture for 24 hours in Example 6;

FIG. 12A is a photograph presenting a state of cells after cell culture for 4 hours in Comparative Example 3;

FIG. 12B is a photograph presenting a state of cells after cell culture for 24 hours in Comparative Example 3;

FIG. 13 is an exemplary view illustrating an example of a three-dimensional culture structure of the present invention;

FIG. 14 is an exemplary view illustrating an example of disposition of a cell, a cell support material, and bioaffinity particles in a three-dimensional culture structure of the present invention;

FIG. 15 is an exemplary view illustrating another example of disposition of a cell, a cell support material, and bioaffinity particles in a three-dimensional culture structure of the present invention;

FIG. 16A is an exemplary view illustrating an example of a step of obtaining a gelatin aqueous solution;

FIG. 16B is an exemplary view illustrating an example of a step of obtaining a cloudy liquid from the gelatin aqueous solution of FIG. 16A;

FIG. 16C is an exemplary view illustrating an example of a step of obtaining gelatin particles from the cloudy liquid of FIG. 16B;

FIG. 16D is an exemplary view illustrating an example of a step of obtaining powder of gelating particles by refining the gelatin particles of FIG. 16C; and

FIG. 17 is an exemplary view illustrating an example of gelation of a cell support material precursor.

### DESCRIPTION OF THE EMBODIMENTS

The present invention has an object to provide a three-dimensional culture structure that is excellent in cell adhesion and cell stretching and can be produced efficiently.

The present invention can provide a three-dimensional culture structure that is excellent in cell adhesion and cell stretching and can be produced efficiently.

### (Three-Dimensional Culture Structure)

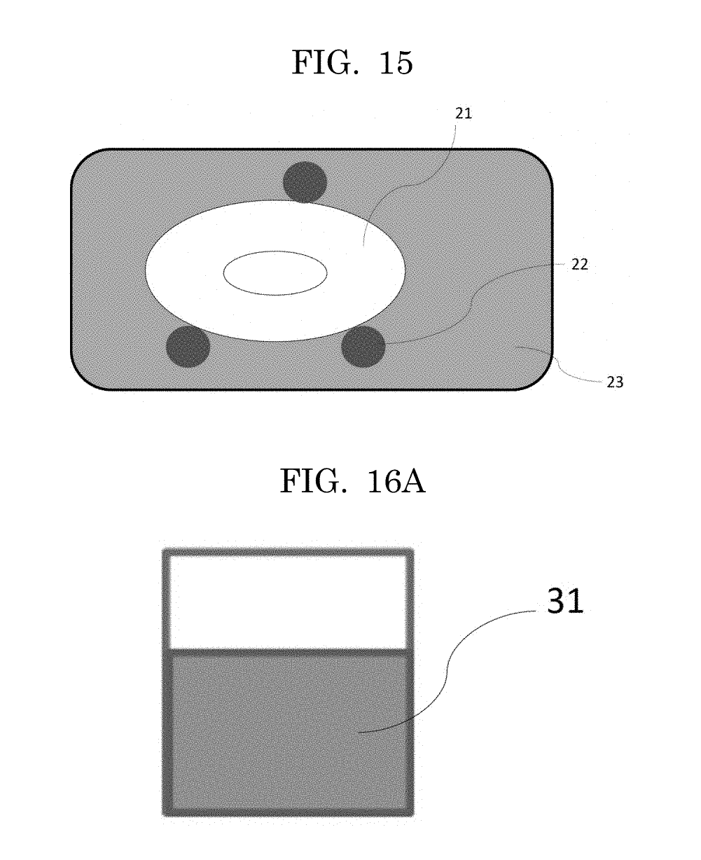

The three-dimensional culture structure of the present invention includes cells, a cell support material configured to support the cells, and bioaffinity particles, and further includes other components as needed. FIG. 14 and FIG. 15 are exemplary views illustrating examples of disposition of a cell, the cell support material, and the bioaffinity particles in the three-dimensional culture structure of the present invention.

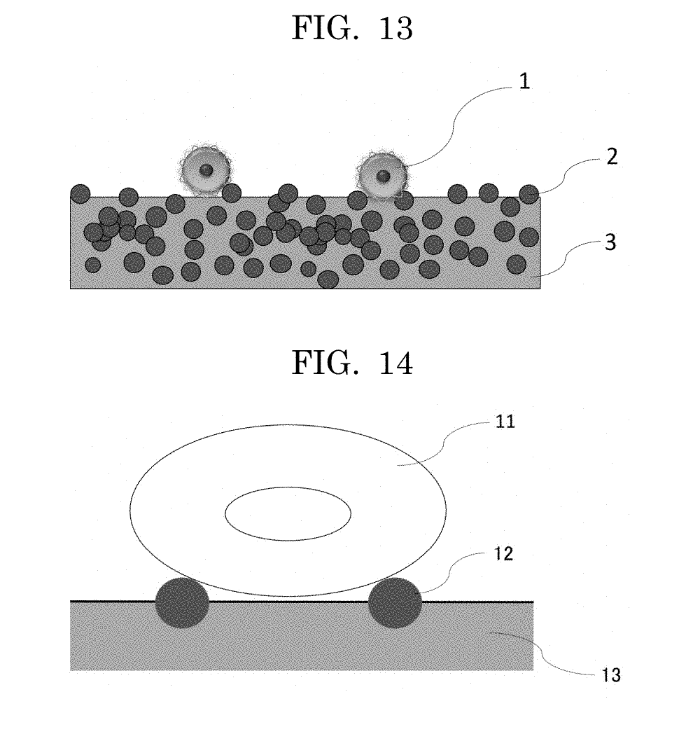

FIG. 13 is an exemplary view illustrating an example of the three-dimensional culture structure of the present invention. As illustrated in FIG. 13, the bioaffinity particles 2 are present in the cell support material 3, and the bioaffinity particles 2 are exposed from at least part of the surface of the cell support material 3. By contact of the cells 1 with the exposed bioaffinity particles 2, adhesion and stretching of the cells can be better promoted.

### <Bioaffinity Particles>

The bioaffinity particles are not particularly limited and may be appropriately selected depending on the intended purpose so long as the bioaffinity particles have a point of adhesion with a living body such as a cell. Examples of the bioaffinity particles include gelatin particles, polylactic acid particles, polystyrene particles, and silica particles. The particulate gelatin particles can improve adhesiveness of cells to the three-dimensional culture structure. Compared with non-particulate gelatin, the particulate gelatin particles can exist in the three-dimensional culture structure for a long term without being degraded by the cells. Therefore, the particulate gelatin particles are advantageous in that the particulate gelatin particles improve adhesiveness of the cells and are utilized as a source of nutrients for the cells for a long term. Moreover, the particulate gelatin particles can suppress a three-dimensional culture structure composition liquid to a low viscosity during production of the three-dimensional culture structure. This facilitates production of the three-dimensional culture structure.

Polymers such as non-particulate gelatin have a problem that nozzles are likely to be clogged by the polymers when the polymers are as is discharged by an inkjet method because the polymers tend to form a film. However, with the particulate bioaffinity material of the present invention, what is discharged can be a solution (ink) in which particles are scattered, and nozzle clogging can be prevented. Moreover, particulate gelatin is present in the cell support material in the form of scattered separate particles. This makes it possible to prevent the viscosity of the cell support material from being increased by mixing the cell support material with gelatin particles.

Furthermore, non-particulate gelatin is brittle in a culture temperature range. Therefore, when one layer of a multi-layer structure contains a solution in which gelatin or a gelatin solution is mixed, there is a problem that the structure easily collapses. However, with particulate gelatin, it is possible to form a structure without degradation of the strength of the structure in the culture temperature range. That is, it is possible to form a structure without shape collapse, even when the structure to be formed is a multi-layer structure in which many layers are laminated in the vertical direction.

The point of adhesion is not particularly limited and may be appropriately selected depending on the intended purpose. Examples of the point of adhesion include a cell adhesion signal.

Examples of the cell adhesion signal include an arginine-glycine-aspartic acid sequence (RGD sequence), a serine-alanine-serine sequence (SAS sequence), a CS1 sequence, a SC5 sequence, a REDV sequence, a GPEILDVPST sequence, a YIGSR sequence, a RNIAEIIKDI sequence, an F-9 peptide, an IKVAV sequence, and a PDSGR sequence.

Adhesion at the point of adhesion means that cells are bonded with the sequence possessed by a material forming a scaffold. The material forming the scaffold may contain the sequence in the own structure or may be provided with the sequence forming the point of adhesion by a treatment such as coating and chemical modification. Adhesion of cells and the sequence with each other is needed for cell growth and cell division for forming the three-dimensional structure.

The bioaffinity particles are not particularly limited, and properties of the bioaffinity particles such as shape may be appropriately selected depending on the intended purpose.

Examples of the shape of the bioaffinity particles include a spherical shape, a linear shape, and an irregular shape. One of these shapes may be used alone or two or more of these shapes may be used in combination.

It is preferable that the bioaffinity particles be exposed from at least part of the surface of the cell support material, more preferably protruded from the cell support material.

“Exposed” means that internally present bioaffinity particles appear on the surface of the three-dimensional culture structure when seen in a plan view.

Whether the bioaffinity particles are exposed can be confirmed with, for example, a scanning electron microscope (SEM) or an atomic force microscope (AFM).

Whether the bioaffinity particles are protruded can be confirmed by observation of a thickness-wise cross-section of the three-dimensional culture structure with, for example, a scanning electron microscope (SEM) or an atomic force microscope (AFM).

In order to increase the bioaffinity particles that are exposed from the surface (i.e., in order to broaden the area to be occupied by the bioaffinity particles), for example, it is possible to increase the concentration of gelatin in the solution (ink) Increase in the number of bioaffinity particles in the solution inevitably causes increase in the number of particles to be exposed from the surface, along with a shorter distance between the bioaffinity particles on the surface.

The distance between the bioaffinity particles in the horizontal direction is preferably shorter than at least the size of one cell used. In this state, at least 2 or more bioaffinity particles adhere to each cell. Therefore, cells can adhere to the cell support material while stretching on the cell support material. It is preferable that the cells 11 adhere to the cell support material 13 via the bioaffinity particles 12 (FIG. 14). It is preferable that the bioaffinity particles 22 adhere to the circumference of the cell 21 on the cell support material 23 (FIG. 15).

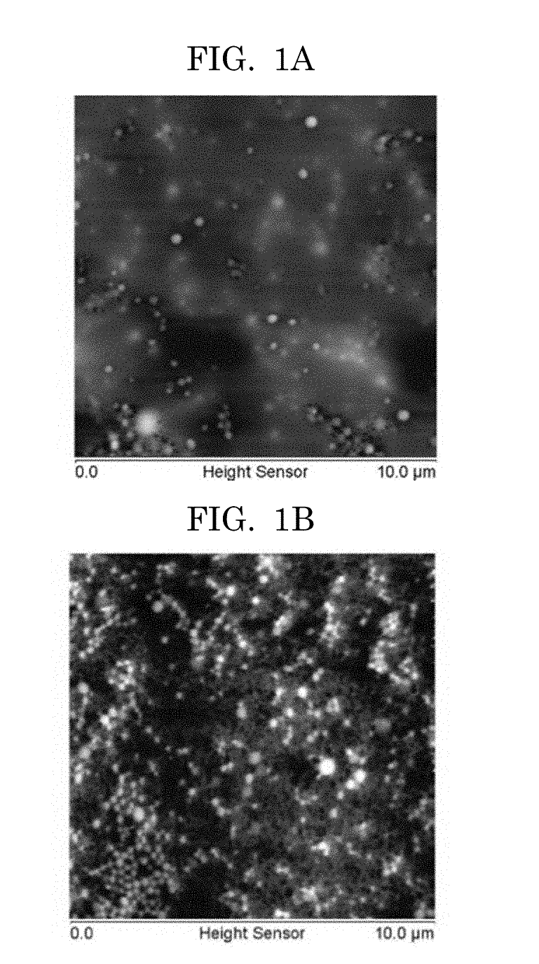

When the surface area occupation rate at which the bioaffinity particles are exposed is 1% or greater of the entire surface of the three-dimensional culture structure, cells can adhere. For efficient stretching of the cells, the surface area occupation rate is preferably 20% or greater and particularly preferably 50% or greater. FIG. 1A, FIG. 1B, and FIG. 1C present atomic force microscope (AFM) photographs of the bioaffinity particles exposed at the surface area occupation rate of 1%, 18%, and from 50% through 58%, respectively.

The occupation rate can be calculated by analyzing an image captured by a scanning electron microscope (SEM) or an atomic force microscope (AFM) with IMAGE J (software).

In another mode, it is also preferable that the bioaffinity particles be dispersed in the cell support material.

The dispersion refers to a phenomenon that a substance having a continuous homogeneous phase contain another substance in a scattered state in the form of particles.

The cumulant diameter of the bioaffinity particles is not particularly limited, may be appropriately selected depending on the intended purpose, and is preferably 0.1 μm or greater but 1.0 μm or less, more preferably 0.25 μm or greater but 0.7 μm or less, and particularly preferably 0.30 μm or greater but 0.70 μm or less. The cumulant diameter can be measured with a thick-system particle diameter analyzer (product name: FPAR-1000, available from Otsuka Electronics Co., Ltd.), using a sample liquid obtained in Sample liquid preparation example described below, under measurement conditions described below. The cumulant diameter of the bioaffinity particles is a particle diameter of the bioaffinity particles in a swollen state. The particle size distribution of the bioaffinity particles can be measured with a particle size distribution meter (product name: UPA150, available from Nikkiso Co., Ltd.).

### —Sample Liquid Preparation Example—

The bioaffinity particles are dispersed at a concentration of 0.5% by mass in pure water obtained with a pure water producing apparatus (product name: GSH-2000, available from ADVANTEC Co., Ltd.). The amount of the liquid for measurement is 5 mL. The sample liquid can be prepared by stirring a 20 mm rotor by a stirrer at 200 rpm for about 1 day.

### —Measurement Conditions—

Solvent: water (with a refractive index of 1.3314 and a viscosity at 25 degrees C. of 0.884 mPa·s (cP), optimum light volume appropriately set with a ND filter)

Measuring probe: a probe for thick systems

Measurement routine: measurement at 25 degrees C. for 180 seconds, then measurement at 25 degrees C. for 600 seconds (monitoring of particle diameter change during gradual liquid temperature change from 25 degrees C. to 35 degrees C. upon a setting change at the main instrument side to 35 degrees C.), and then measurement at 35 degrees C. for 180 seconds

The bioaffinity particles may be an appropriately synthesized product or a commercially available product.

When the bioaffinity particles are gelatin particles, examples of commercially available products of gelatin, which is the material of the gelatin particles, include product name APH-250 (available from Nitta Gelatin Inc.), gelatin (available from Wako Pure Chemical Industries, Ltd.), gelatin (available from Nacalai Tesque, Inc.), and MEDIGELATIN (available from Nippi, Inc.).

Examples of commercially available products of the polylactic acid particles include product name: PLA PARTICLES (available from Corefront Corporation).

Examples of commercially available products of the polystyrene particles include product name MICROMER-RED F (available from Corefront Corporation).

Examples of commercially available products of the silica particles include product name SICASTAR-RED F (available from Corefront Corporation).

It is preferable that the bioaffinity particles be cross-linked by a cross-linking agent in the structure of the bioaffinity particles. When gelatin particles are used as the bioaffinity particles, it is preferable to cross-link gelatin by a cross-linking agent. By cross-linking by a cross-linking agent, the cumulant diameter of the bioaffinity particles can be suppressed and proliferation of cells can be promoted on the three-dimensional culture structure containing the bioaffinity particles.

The cross-linking agent is not particularly limited and may be appropriately selected depending on the intended purpose. Examples of the cross-linking agent include: aldehydes such as glutaraldehyde and formaldehyde; glycidyl ethers such as ethylene propylene diglycidyl ether, glycerol polyglycidyl ether, diglycerol polyglycidyl ether, sorbitol polyglycidyl ether, and ethylene glycol diglycidyl ether; isocyanates such as hexamethylene diisocyanate, α-tolidine isocyanate, tolylene diisocyanate, naphthylene-1,5-diisocyanate, 4,4-diphenylmethane diisocyanate, and triphenylmethane-4,4,4-triisocyanate; calcium gluconate. One of these cross-linking agents may be used alone or two or more of these cross-linking agents may be used in combination. Among these cross-linking agents, aldehydes are preferable, and glutaraldehyde is more preferable.

When the bioaffinity particles are gelatin particles, the content of the cross-linking agent is preferably 1% by mass or greater but 20% by mass or less and more preferably 2% by mass or greater but 10% by mass or less of the total amount of gelatin. When the content of the cross-linking agent is 1% by mass or greater but 20% by mass or less, the cumulant diameter of the bioaffinity particles can be suppressed and proliferation of cells can be promoted on the three-dimensional culture structure containing the bioaffinity particles.

The content of the bioaffinity particles is preferably 0.5% by mass or greater but 10% by mass or less, more preferably 0.5% by mass or greater but 5% by mass or less, and particularly preferably 0.5% by mass or greater but 2% by mass or less of the total amount of the three-dimensional culture structure. When the content of the bioaffinity particles is 0.5% by mass or greater but 10% by mass or less, cells can adhere to the three-dimensional culture structure sufficiently, and cell proliferation can be promoted.

### [Method for Producing Bioaffinity Particles]

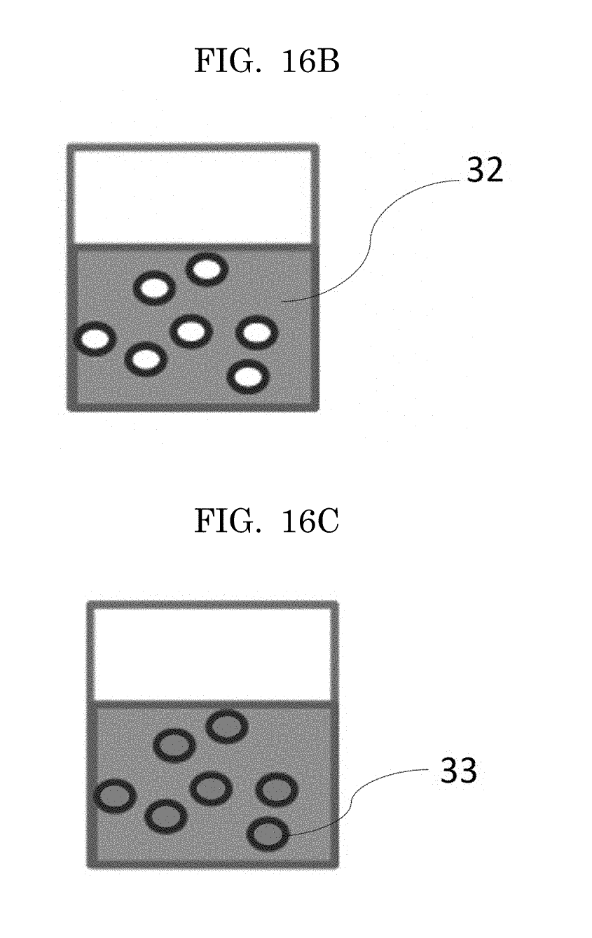

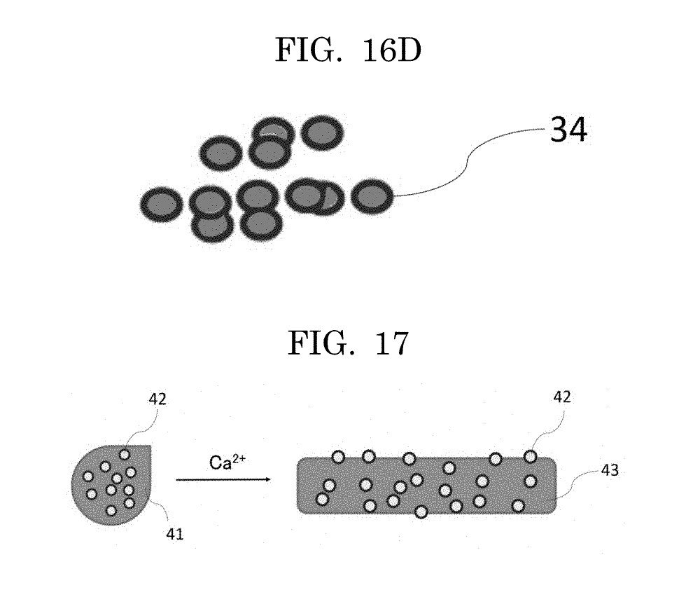

The bioaffinity particles can be produced in the manner described below, where an example of gelatin particles is described with reference to FIG. 16A to FIG. 16D.

As bioaffinity particles, gelatin is mixed with water to have a concentration of 2% by mass and dissolved in the water in a water bath of 60 degrees C., to obtain a 2% by mass gelatin aqueous solution 31 (FIG. 16A). Next, the 2% by mass gelatin aqueous solution (40 mL) heated to 40 degrees C. is poured in a 200 mL beaker and stirred. Subsequently, acetone (60 g) is added in one breath, to obtain a cloudy liquid 32 (coacervation, FIG. 16B).

To the cloudy liquid 32, a glutaraldehyde aqueous solution having a concentration of 24% by mass or greater but 26% by mass or less is added as a cross-linking agent in an amount of, for example, 160 μL (in a glutaraldehyde content of 5% by mass of the total amount of gelatin). The resultant is retained in a water bath of 60 degrees C. for 30 minutes under stirring at about 300 rpm. The cloudy liquid gradually turns to a cream color and becomes able to form gelatin particles 33 (FIG. 16C). Next, the resultant is returned to room temperature (25 degrees C.). To the resultant, acetone (100 g) is added to aggregate and precipitate gelatin particles, to obtain a precipitate.

Removal of the supernatant and washing with acetone are repeated a few times, to remove water and unreacted part of the cross-linking agent from the obtained precipitate. The resultant is subjected to filtration as needed. Subsequently, the precipitate is dried on a hot plate at 60 degrees C. and dried at reduced pressure for 1 hour on the hot plate at 50 degrees C., to obtain powder 34 of gelatin particles (bioaffinity particles) (FIG. 16D).

### <Cell Support Material (Intercellular Distance Adjusting Material)>

The intercellular distance adjusting material (hereinafter may also be referred to as “cell support material”) is not particularly limited and may be appropriately selected depending on the intended purpose, so long as the intercellular distance adjusting material can support cells. However, an intercellular distance adjusting material having biocompatibility is preferable.

The cell support material is not particularly limited and may be appropriately selected depending on the intended purpose. Examples of the cell support material include polysaccharides, amphoteric gels, and protein gels (e.g., fibrin glue). One of these cell support materials may be used alone or two or more of these cell support materials may be used in combination. Among these cell support materials, polysaccharides are preferable.

Among polysaccharides, for example, gelatinous polysaccharides are preferable.

The gelatinous polysaccharides are not particularly limited and may be appropriately selected depending on the intended purpose.

Examples of the gelatinous polysaccharides include calcium alginate, gellan gum, agarose, guar gum, xanthan gum, carrageenan, pectin, locust bean gum, Tamarind gum, diutan gum, and carboxymethyl cellulose. Among these gelatinous polysaccharides, calcium alginate is preferable. Calcium alginate is a salt in which calcium ion is bonded with carboxyl group of alginic acid. Calcium ion is divalent. Hence, calcium ion is bonded (ionically cross-linked) with 2 carboxyl groups in a manner to bridge the 2 carboxyl groups, to thicken the viscosity. In this way, calcium alginate can form a three-dimensional culture structure. The cell support material can support (hold) cells in predetermined regions. Therefore, the cell support material can adjust the distance between the cells.

The content of the cell support material is preferably 10% by mass or greater but less than 100% by mass of the total amount of the three-dimensional culture structure. When the content is 10% by mass or greater but less than 100%, the strength of the cell support material can be suitable as a scaffold on which cells can adhere and stretch.

### [Method for Measuring Bioaffinity Particles and Cell Support Material in Three-Dimensional Culture Structure]

Examples of the method for measuring the bioaffinity particles and the cell support material in the three-dimensional culture structure include: a method of measuring the bioaffinity particles and the cell support material based on peak intensities obtained by GC-MS measurement; a method for measuring the bioaffinity particles and the cell support material based on peak intensities obtained by molecular weight distribution measurement by gel permeation chromatography (GPC); and a method for measuring the bioaffinity particles and the cell support material based on integral values obtained by ¹H NMR measurement.

### <Cells>

The kind, etc. of the cells are not particularly limited and may be appropriately selected depending on the intended purpose. Taxonomically irrespectively, all kinds of cells such as eukaryotic cells, prokaryotic cells, multicellular organisms' cells, and unicellular organisms' cells may be used.

Examples of the eukaryotic cells include animal cells, insect cells, plant cells, and mycoses. One of these eukaryotic cells may be used alone or two or more of these eukaryotic cells may be used in combination. Among these eukaryotic cells, animal cells are preferable. For formation of cell aggregates by the cells, adherent cells having a cell adhesiveness of a level at which cells adhere with each other and are not isolated without a physicochemical treatment are more preferable.

The adherent cells are not particularly limited and may be appropriately selected depending on the intended purpose. Examples of the adherent cells include differentiated cells and undifferentiated cells.

Examples of the differentiated cells include: hepatocyte, which is the parenchymal cell of liver; stellate cell; Kupffer cell; endothelial cells such as vascular endothelial cell, sinusoidal endothelial cell, and corneal endothelial cell; fibroblast; osteoblast; osteoclast; periodontal ligament-derived cell; epidermal cell such as epidermal keratinocyte; epithelial cells such as tracheal epithelial cell, intestinal epithelial cell, cervical epithelial cell, and corneal epithelial cell; mammary glandular cell; pericyte; muscle cells such as smooth muscle cell and cardiac muscle cell; kidney cell; pancreas islet cell; nerve cells such as peripheral nerve cell and optic nerve cell; cartilage cell; and bone cell. The adherent cells may be primary cells directly picked from tissues and organs or may be cells obtained by subculturing primary cells by a few times.

The undifferentiated cells are not particularly limited and may be appropriately selected depending on the intended purpose. Examples of the undifferentiated cells include: pluripotent stem cells such as undifferentiated embryonic stem cell and mesenchymal stem cell having multipotency; unipotent stem cells such as vascular endothelial progenitor cell having unipotency; and iPS cell.

Examples of the prokaryotic cells include eubacteria and archaea. The viscosity of the three-dimensional culture structure is not particularly limited, may be appropriately selected depending on the intended purpose, and is preferably 200 mPa·s or greater but 500 mPa·s or less and more preferably 300 mPa·s or greater but 400 mPa·s or less. When the viscosity of the three-dimensional culture structure is 200 mPa·s or greater but 500 mPa·s or less, close adhesiveness, proliferation, and stretching of cells can be improved.

The viscosity of the three-dimensional culture structure can be measured under the conditions described below.

### [Viscosity Measurement Conditions]

Measuring instrument: MCR-301 (available from Anton Paar Japan K.K.)

Cone: CP50-1

Temperature: 25 degrees C.

Shear velocity: 120 (l/s)

### (Method for Producing Three-Dimensional Culture Structure)

The method for producing a three-dimensional culture structure of the present invention includes a layer forming step, a cell support material precursor gelating aqueous solution applying step, and a cell layer forming material applying step, and further includes other steps as needed.

The method for producing a three-dimensional culture structure of the present invention can produce the three-dimensional culture structure of the present invention suitably.

### <Layer Forming Step>

The layer forming step is a step of forming a cell support material precursor aqueous solution layer on a base material, the cell support material precursor aqueous solution layer being formed of a cell support material precursor aqueous solution.

### —Cell Support Material Precursor Aqueous Solution Layer—

The cell support material precursor aqueous solution layer contains a cell support material precursor aqueous solution (culture ink) and further contains any other aqueous solutions as needed.

The cell support material precursor aqueous solution (culture ink) contains bioaffinity particles and a cell support material precursor, and further contains other components as needed.

### ——Bioaffinity Particles——

As the bioaffinity particles, the same bioaffinity particles as the bioaffinity particles in the three-dimensional culture structure of the present invention may be used.

### ——Cell Support Material Precursor——

The cell support material precursor is not particularly limited and may be appropriately selected depending on the intended purpose. Examples of the cell support material precursor include: living body-derived polymers such as collagen, elastin, and gelatin; metal salts of polysaccharide compounds, such as alginic acid and gellan gum; and synthetic polymers such as polylactic acid. One of these cell support material precursors may be used alone or two or more of these cell support material precursors may be used in combination. Among these cell support material precursors, metal salts of polysaccharide compounds are preferable, and alginates are more preferable.

The alginates are not particularly limited and may be appropriately selected depending on the intended purpose. Examples of the alginates include sodium alginate, potassium alginate, and ammonium alginate. One of these alginates may be used alone or two or more of these alginates may be used in combination. Among these alginates, sodium alginate is preferable.

It is preferable to form the cell support material precursor aqueous solution layer by discharging the cell support material precursor aqueous solution onto the base material.

Discharging of the cell support material precursor aqueous solution onto the base material is not particularly limited and may be appropriately selected depending on the intended purpose. However, an inkjet method is preferable.

Examples of the method for discharging the cell support material precursor aqueous solution include a so-called piezo method (see Japanese Examined Patent Publication No. 02-51734) using a piezoelectric element as a pressure generating unit to pressurize a liquid in a liquid flow path to deform a vibration plate constituting a wall surface of the liquid flow path and change the internal capacity of the liquid flow path to discharge liquid droplets, a so-called thermal method (see Japanese Examined Patent Publication No. 61-59911) using a heating resistor to heat a liquid in a liquid flow path and generate bubbles, and an electrostatic method (see Japanese Unexamined Patent Application Publication No. 06-71882) using a vibration plate constituting a wall surface of a liquid flow path and an electrode disposed counter to the vibration plate to deform the vibration plate by the effect of an electrostatic force generated between the vibration plate and the electrode and change the internal capacity of the liquid flow path to discharge liquid droplets.

The size of the liquid droplets of the cell support material precursor aqueous solution to be discharged is preferably 3 pL or greater but 40 pL or less. A discharging/jetting speed of the liquid droplets of the cell support material precursor aqueous solution is preferably 5 m/s or higher but 20 m/s or lower. A driving frequency for discharging the liquid droplets of the cell support material precursor aqueous solution is preferably 1 kHz or higher.

The viscosity of the cell support material precursor aqueous solution is not particularly limited, may be appropriately selected depending on the intended purpose, and is preferably 20 mPa·s or less and more preferably 15 mPa·s or less. When the viscosity of the cell support material precursor aqueous solution is 20 mPa·s or less, the cell support material precursor aqueous solution can easily form a liquid layer on the base material and can be improved in discharging stability when an inkjet method is used.

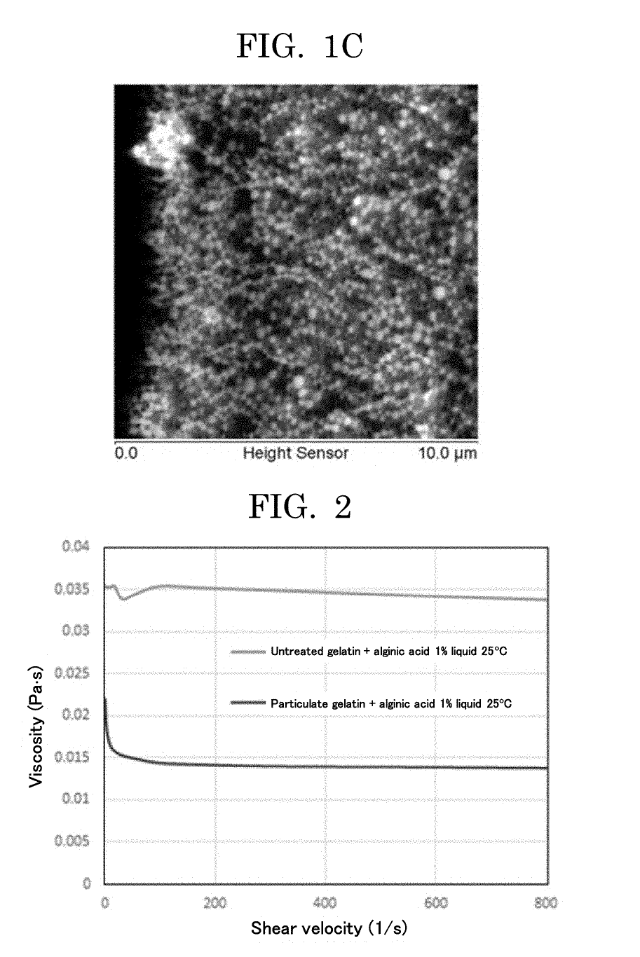

It is possible to adjust the viscosity of the cell support material precursor aqueous solution to a viscosity suitable for an inkjet method by making a bioaffinity material into particulate bioaffinity particles. FIG. 2 is a graph plotting viscosity of a liquid containing gelatin particles, which are the bioaffinity particles and viscosity of a liquid containing gelatin, which is the bioaffinity material. As plotted in FIG. 2, it can be understood that the viscosity of the liquid containing a 1% by mass particulate gelatin and 1% by mass sodium alginate is lower than the viscosity of the liquid containing a 1% by mass untreated gelatin and 1% by mass sodium alginate, and that the viscosity of the former is suitable for an inkjet method.

The untreated gelatin that is mixed with sodium alginate and gelated dissolves and drains out at a cell culture temperature, and cannot maintain the cell support strength but collapses. Hence, the untreated gelatin cannot be used for culture of cells. On the other hand, the particulate gelatin does not undergo temperature-dependent shape change and can maintain the cell support strength. Hence, the particulate gelatin can suitably culture cells without collapsing during culture of cells.

The average thickness of the cell support material precursor aqueous solution layer is not particularly limited, may be appropriately selected depending on the intended purpose, and is preferably 3 μm or greater but 200 μm or less and more preferably 10 μm or greater but 100 μm or less. When the average thickness of the cell support material precursor aqueous solution layer is 3 μm or greater but 200 μm or less, proliferation of cells can be promoted suitably. The average thickness of the cell support material precursor aqueous solution layer can be measured according to a known method.

The volume average particle diameter of the bioaffinity particles is not particularly limited, may be appropriately selected depending on the intended purpose, and is preferably 0.1 μm or greater but 5 μm or less and more preferably 0.1 μm or greater but 3 μm or less. When the volume average particle diameter of the bioaffinity particles is 0.1 μm or greater but 5 μm or less, the bioaffinity particles can suppress the viscosity and can adjust the viscosity to a viscosity suitable for discharging by an inkjet method. The volume average particle diameter of the bioaffinity particles can be measured in the same manner as the method for measuring the cumulant diameter of the bioaffinity particles.

### —Base Material—

The size, shape, structure, material, etc. of the base material are not particularly limited and may be appropriately selected depending on the intended purpose so long as the base material does not inhibit the activity and proliferation of cells.

The size of the base material is not particularly limited and may be appropriately selected depending on the intended purpose.

The shape of the base material is not particularly limited and may be appropriately selected depending on the intended purpose. Examples of the shape of the base material include: three-dimensional shapes such as dishes, multi-plates, flasks, and cell inserts; and flat membrane shapes.

The structure of the base material is not particularly limited and may be appropriately selected depending on the intended purpose. Examples of the structure of the base material include a porous structure, a mesh structure, a concave-convex structure, and a honeycomb structure.

Examples of the material of the base material include organic materials and inorganic materials. One of these materials may be used alone or two or more of these materials may be used in combination.

The organic materials are not particularly limited and may be appropriately selected depending on the intended purpose. Examples of the organic materials include polyethylene terephthalate (PET), polystyrene (PS), polycarbonate (PC), triacetyl cellulose (TAC), polyimide (PI), nylon (Ny), low-density polyethylene (LDPE), middle-density polyethylene (MDPE), vinyl chloride, vinylidene chloride, polyphenylene sulfide, polyether sulfone, polyethylene naphthalate, polypropylene, acrylic-based materials such as urethane acrylate, and cellulose

The inorganic materials are not particularly limited and may be appropriately selected depending on the intended purpose. Examples of the inorganic materials include glass and ceramics.

### <Cell Support Material Precursor Gelating Aqueous Solution Applying Step>

The cell support material precursor gelating aqueous solution applying step is a step of applying a cell support material precursor gelating aqueous solution, which is configured to gelate the cell support material precursor upon contact with the cell support material precursor aqueous solution, on the cell support material precursor aqueous solution layer.

When the cell support material precursor gelating aqueous solution is applied on the cell support material precursor aqueous solution layer, the cell support material precursor in the cell support material precursor aqueous solution undergoes a reaction with a cell support material precursor gelating polymer and is ionically cross-linked and thickened, to make it possible to form a three-dimensional culture structure.

### —Cell Support Material Precursor Gelating Aqueous Solution—

The cell support material precursor gelating aqueous solution contains a cell support material precursor gelating polymer that gelates the cell support material precursor upon contact with the cell support material precursor aqueous solution, and further contains other components as needed.

The cell support material precursor gelating aqueous solution is not particularly limited and may be appropriately selected depending on the intended purpose. Preferable examples of the cell support material precursor gelating aqueous solution include a calcium chloride aqueous solution, a chitosan aqueous solution, and a chitin aqueous solution.

### ——Cell Support Material Precursor Gelating Polymer——

The cell support material precursor gelating polymer is not particularly limited and may be appropriately selected depending on the intended purpose so long as the cell support material precursor gelating polymer can gelate the cell support material precursor upon contact with the cell support material precursor aqueous solution. Examples of the cell support material precursor gelating polymer include polysaccharides, polyvalent metal salts, fibrinogen, thrombin, fibronectin, laminin, recombinant peptide, chitosan, and chitin. One of these cell support material precursor gelating polymers may be used alone or two or more of these cell support material precursor gelating polymers may be used in combination. Among these cell support material precursor gelating polymers, polyvalent metal salts, chitosan, and chitin are preferable, calcium chloride, chitosan, and chitin are more preferable, and calcium chloride is particularly preferable.

FIG. 17 is an exemplary view illustrating an example of gelation of a cell support material precursor. As illustrated in FIG. 17, when a cell support material precursor gelating polymer such as calcium ion derived from, for example, calcium chloride is supplied to a cell support material precursor 41 containing bioaffinity particles 42, a cell support material 43 containing the bioaffinity particles 42 can be formed.

It is preferable to apply the cell support material precursor gelating aqueous solution on the cell support material precursor aqueous solution layer in the cell support material precursor gelating aqueous solution applying step, by discharging the cell support material precursor gelating aqueous solution onto the cell support material precursor aqueous solution layer.

Discharging of the cell support material precursor gelating aqueous solution onto the cell support material precursor aqueous solution layer is not particularly limited and may be appropriately selected depending on the intended purpose. However, an inkjet method is preferable.

As the inkjet method, the same inkjet method as the inkjet method in the layer forming step may be employed.

### <Cell Layer Forming Material Applying Step>

The cell layer forming material applying step is a step of applying a cell layer forming material containing cells on a three-dimensional culture structure formed through the layer forming step and the cell support material precursor gelating aqueous solution applying step.

As the cells, the same cells as the cells in the three-dimensional culture structure may be used.

### —Method for Applying Cell Layer Forming Material—

The method for applying the cell layer forming material is not particularly limited and may be appropriately selected depending on the intended purpose. Examples of the method include an inkjet method, a dispenser method, a pipette method, and an aspirator method. One of these methods may be used alone or two or more of these methods may be used in combination. Among these methods, an inkjet method is preferable because the inkjet method can realize precise, complicated cell placement at a single cell level.

The inkjet method is not particularly limited, and a known method may be used. For performing the inkjet method, a known inkjet discharging apparatus can be suitably used as a cell layer forming material applying unit.

The volume average particle diameter of the cells in a free state is preferably 100 μm or less, more preferably 50 μm or less, and particularly preferably 20 μm or less. When the volume average particle diameter of the cells is 100 μm or less, the cells can be suitably used in an inkjet method.

The volume average particle diameter of the cells can be measured according to the measuring method described below.

In an incubator (product name: KM-CC17RU2, available from Panasonic Corporation, 37 degrees C., 5% CO₂ environment), cells are cultured in a Dulbecco's modified Eagle's medium (available from Wako Pure Chemical Industries, Ltd., hereinafter may also be referred to as “D-MEM”) containing a 1% by mass antibiotic (ANTIBIOTIC-ANTIMYCOTIC MIXED STOCK SOLUTION (100×), available from Wako Pure Chemical Industries, Ltd.). Subsequently, a 10% by mass fetal bovine serum (hereinafter may also be referred to as “FBS”) and the aforementioned medium are removed from a 100 mm dish, using an aspirator (product name: VACUSIP, available from INTEGRA Inc.). A phosphate buffered saline (available from Wako Pure Chemical Industries, Ltd., hereinafter may also be referred to as “PBS (−)”) (3 mL) is added into the dish, and then removed by suctioning with the aspirator, to wash the surface of the cells. After the washing operation using the PBS (−) is repeated 3 times, a 0.1% by mass trypsin solution (Trypsin, from Porcine Pancreas, available from Wako Pure Chemical Industries, Ltd.) (3 mL) is added into the dish and the resultant is heated in the incubator for 5 minutes, to strip the cells from the dish. After it is confirmed with a phase-contrast microscope that the cells are stripped, D-MEM (4 mL) containing 10% by mass FBS and a 1% by mass antibiotic is added into the dish, to deactivate the trypsin. The content in the dish is moved into a centrifuge tube and subjected to centrifugation (with product name: H-19FM, available from KOKUSAN Co., Ltd., at 1.2×10³ rpm (234G), for 5 min, at 5 degrees C.), and the supernatant is removed with the aspirator. After the removal, D-MEM (1 mL) containing 10% by mass FBS and a 1% by mass antibiotic is added into the centrifuge tube, and pipetting is performed gently to disperse the cells. Ten microliters is taken out from the obtained cell layer forming material into an Eppendorf tube, a 0.4% by mass trypan blue stain (10 μL) is added into the tube, and pipetting is performed to stain the cells. Ten microliters is taken out from the stained cell layer forming material and put on a PMMA-made plastic slide. The cells can be measured with an automatic cell counter (product name: COUNTESS AUTOMATED CELL COUNTER, available from Invitrogen Co., Ltd.). The number of cells and the cell surviving rate can also be obtained by the same measuring method. The cells have an approximately spherical shape when the cells are in a free state. Hence, the volume average particle diameter of the cells can be measured.

The number of cells in the cell layer forming material is not particularly limited, may be appropriately selected depending on the intended purpose, and is preferably 5×10⁵ cells/mL or more but 5×10⁸ cells/mL or less and more preferably 5×10⁵ cells/mL or more but 5×10⁷ cells/mL or less. When the number of cells is 5×10⁵ cells/mL or more but 5×10⁸ cells/mL or less, it can be ensured that the cells are contained in the liquid droplets to be discharged. This is suitable for precise placement of cells. The number of cells can be measured with an automatic cell counter (product name: COUNTESS AUTOMATED CELL COUNTER, available from Invitrogen Co., Ltd.) as in the method for measuring the volume average particle diameter.

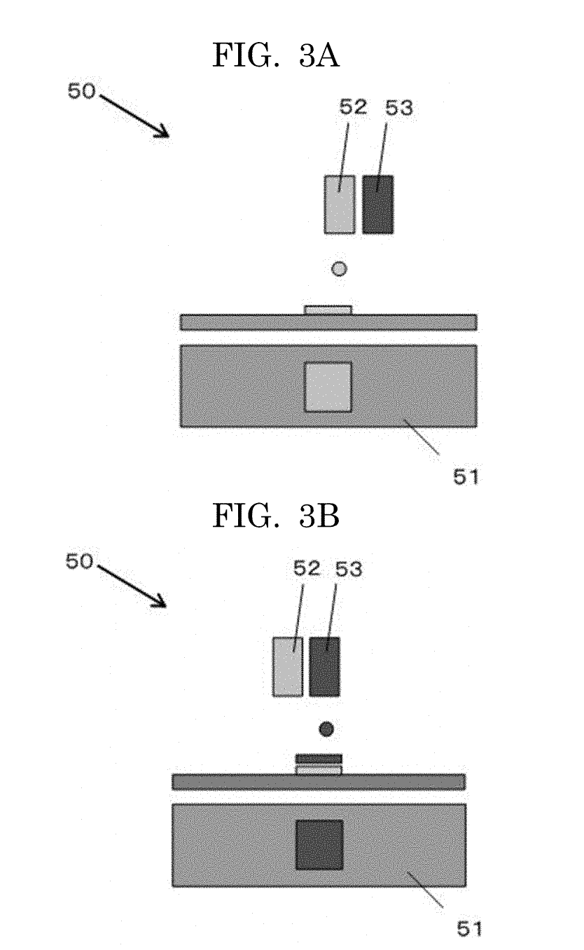

FIG. 3A is a schematic diagram illustrating an example of a producing apparatus configured to produce a three-dimensional culture structure used in the present invention. FIG. 3B is a schematic view illustrating another example of a producing apparatus configured to produce a three-dimensional culture structure used in the present invention. Using a head unit in which inkjet heads 52 and 53 are arranged, the three-dimensional culture structure producing apparatuses of FIG. 3A and FIG. 3B are configured to discharge a cell support material precursor aqueous solution from the inkjet head 52 onto a base material 51 to form a cell support material precursor aqueous solution layer and then discharge a cell support material precursor gelating aqueous solution from the inkjet head 53 onto the cell support material precursor aqueous solution layer to bring the cell support material precursor gelating aqueous solution into contact with the cell support material precursor aqueous solution in the cell support material precursor aqueous solution layer to form a cell support material. Subsequently, the three-dimensional culture structure producing apparatuses can form a cell layer by discharging a cell layer forming material onto the cell support material formed. In this case, the cell layer forming material may be discharged in a state of being contained in the cell support material precursor aqueous solution or in the cell support material precursor gelating aqueous solution, or may be discharged from an inkjet head other than the inkjet heads 52 and 53. It is also possible to produce the three-dimensional culture structure of the present invention by repeating forming the cell support material and the cell layer.

### EXAMPLES

Examples of the present invention and Comparative Examples will be described below. However, the present invention should not be construed as being limited to these Examples.

Cumulant diameter and particle size distribution of gelatin particles, viscosity of a cell support material precursor aqueous solution, surface area occupation rate at which bioaffinity particles were exposed, and presence or absence of protrusion of bioaffinity particles on the surface of a cell support material were measured in the manners described below.

### <Cumulant Diameter of Gelatin Particles>

The cumulant diameter of the gelatin particles was measured with a thick-system particle diameter analyzer (product name: FPAR-1000, available from Otsuka Electronics Co., Ltd.), using a sample liquid obtained in Sample liquid preparation example described below, under measurement conditions described below.

### —Sample Liquid Preparation Example—

Bioaffinity particles were dispersed at a concentration of 0.5% by mass in pure water obtained with a pure water producing apparatus (product name: GSH-2000, available from ADVANTEC Co., Ltd.). The amount of the liquid for measurement was 5 mL. The bioaffinity particles were dispersed by stirring a 20 mm rotor by a stirrer at 200 rpm for about 1 day. In this way the sample liquid was prepared.

### —Measurement Conditions—

Solvent: water (with a refractive index of 1.3314 and a viscosity at 25 degrees C. of 0.884 mPa·s (cP), optimum light volume appropriately set with a ND filter)

Measuring probe: a probe for thick systems

Measurement routine: measurement at 25 degrees C. for 180 seconds, then measurement at 25 degrees C. for 600 seconds (monitoring of particle diameter change during gradual liquid temperature change from 25 degrees C. to 35 degrees C. upon a setting change at the main instrument side to 35 degrees C.), and then measurement at 35 degrees C. for 180 seconds

### <Particle Size Distribution of Gelatin Particles>

The particle size distribution of the gelatin particles was measured with a particle size distribution meter (product name: UPA150, available from Nikkiso Co., Ltd.).

### <Viscosity of Cell Support Material Precursor Aqueous Solution>

The viscosity of the cell support material precursor aqueous solution was measured under the conditions described below.

### [Viscosity Measurement Conditions]

Measuring instrument: MCR-301 (available from Anton Paar Japan K.K.)

Cone: CP50-1

Temperature: 25 degrees C.

Shear velocity: 120 (l/s)

<Surface Area Occupation Rate at which Bioaffinity Particles were Exposed>

The surface area occupation rate was calculated by analyzing an image obtained by an atomic force microscope (AFM) with IMAGE J (software).

### <Presence or Absence of Protrusion of Bioaffinity Particles on Surface of Cell Support Material>

Presence or absence of protrusion of the bioaffinity particles on the surface of a cell support material was confirmed by observation of a thickness-wise cross-section of the three-dimensional culture structure with, a scanning electron microscope.

### Bioaffinity Particle Aqueous Solution Production Example 1

### <Production of Gelatin Particle Aqueous Solution A>

As the material of the bioaffinity particles, gelatin (product name: APH-250, available from Nitta Gelatin Inc.) (0.5 g) was mixed with water (49.5 mL) and dissolved in the water in a water bath of 60 degrees C., to obtain a 1% by mass gelatin aqueous solution. Next, the 1% by mass gelatin aqueous solution (40 mL) heated to 40 degrees C. was poured in a 200 mL beaker and stirred. Subsequently, acetone (60 g) was added in one breath, to obtain a cloudy liquid (coacervation).

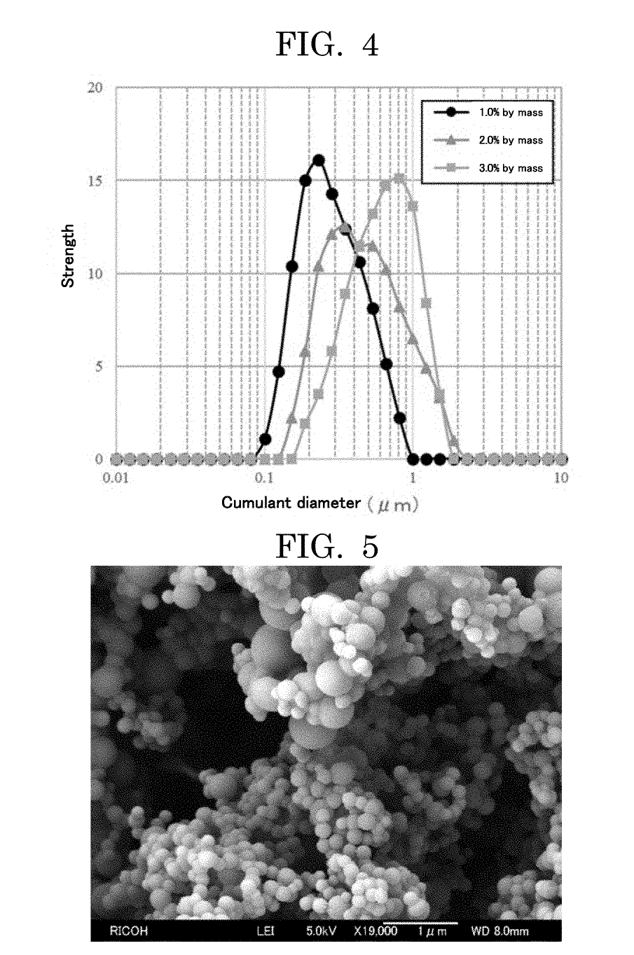

To the cloudy liquid, a glutaraldehyde aqueous solution (available from Tokyo Chemical Industry Co., Ltd.) having a concentration of 24% by mass or greater but 26% by mass or less was added as a cross-linking agent in an amount at which the cross-linking agent would account for 5% by mass of the total amount of gelatin. The resultant was retained in a water bath of 60 degrees C. for 30 minutes under stirring at about 300 rpm. The cloudy liquid gradually turned to a cream color, to be obtained as a 1.0% by mass gelatin particle aqueous solution A (at a cross-linking agent concentration of 5% by mass relative to gelatin). The cumulant diameter of the gelatin particles in the obtained 1.0% by mass gelatin particle aqueous solution A is presented in Table 1 below. The particle size distribution of the gelatin particles is presented in FIG. 4.

### Bioaffinity Particle Aqueous Solution Production Example 2

### <Production of Gelatin Particle Aqueous Solution B>

A 2.0% by mass gelatin particle aqueous solution B (with a cross-linking agent concentration of 5% by mass relative to gelatin and an amount of cross-linking agent of 160 μL) was obtained in the same manner as in Bioaffinity particle aqueous solution production example 1, except that unlike in Bioaffinity particle aqueous solution production example 1, the amount of gelatin was changed from 0.5 g to 1 g, and the amount of water was changed from 49.5 mL to 49 mL. The cumulant diameter of the gelatin particles in the obtained 2.0% by mass gelatin particle aqueous solution B is presented in Table 1 below. The particle size distribution of the gelatin particles is presented in FIG. 4.

Next, the obtained 2.0% by mass gelatin particle aqueous solution B was returned to room temperature (25 degrees C.). To the resultant, acetone (100 g) was added to aggregate and precipitate the gelatin particles, to obtain a precipitate. Removal of the supernatant and washing with acetone were repeated a few times, to remove water and unreacted part of the cross-linking agent from the obtained precipitate. Subsequently, the precipitate was dried on a hot plate at 60 degrees C. and dried at reduced pressure for 1 hour on the hot plate at 50 degrees C., to obtain powder of gelatin particles (at a yield of 70%). A SEM image of the powder of gelatin particles (dry-state gelatin particles) was observed with a scanning electron microscope (instrument name: M-SEM, available from JEOL Ltd.). The result is presented in FIG. 5.

From the result of FIG. 5, the shape of the gelatin particles was spherical. Most of the gelatin particles had a cumulant diameter of 0.1 μm or greater but 0.5 μm or less, but presence of coarse particles having a cumulant diameter of 1 μm or greater was also confirmed. As known from FIG. 4, the cumulant diameter of the gelatin particles in a swollen state was 0.2 μm or greater but 1 μm or less, which was twice as great as the cumulant diameter of the dry-state gelatin particles.

### Bioaffinity Particle Aqueous Solution Production Example 3

### <Production of Gelatin Particle Aqueous Solution C>

A 3.0% by mass gelatin particle aqueous solution C (with a cross-linking agent concentration of 5% by mass relative to gelatin) was obtained in the same manner as in Bioaffinity particle aqueous solution production example 1, except that unlike in Bioaffinity particle aqueous solution production example 1, the amount of gelatin was changed from 0.5 g to 1.5 g, and the amount of water was changed from 49.5 mL to 48.5 mL. The cumulant diameter of the gelatin particles in the obtained 3.0% by mass gelatin particle aqueous solution C is presented in Table 1 below. The particle size distribution of the gelatin particles is presented in FIG. 4.

### Bioaffinity Particle Aqueous Solution Production Example 4

### <Production of Gelatin Particle Aqueous Solution D>

A 4.0% by mass gelatin particle aqueous solution D (with a cross-linking agent concentration of 5% by mass relative to gelatin) was prepared in the same manner as in Bioaffinity particle aqueous solution production example 1, except that unlike in Bioaffinity particle aqueous solution production example 1, the amount of gelatin was changed from 0.5 g to 2 g, and the amount of water was changed from 49.5 mL to 48 mL. As a result, gelatin did not become particles but became a block-state. Because gelatin did not become particles, it was impossible to measure cumulant diameter and particle size distribution of gelatin particles.

### Bioaffinity Particle Aqueous Solution Production Example 5

### <Production of Gelatin Particle Aqueous Solution E>

A 5.0% by mass gelatin particle aqueous solution E (with a cross-linking agent concentration of 5% by mass relative to gelatin) was prepared in the same manner as in Bioaffinity particle aqueous solution production example 1, except that unlike in Bioaffinity particle aqueous solution production example 1, the amount of gelatin was changed from 0.5 g to 2.5 g, and the amount of water was changed from 49.5 mL to 47.5 mL. As a result, gelatin did not become particles but became a block-state. Because gelatin did not become particles, it was impossible to measure cumulant diameter and particle size distribution of gelatin particles.

TABLE 1

| Gelatin | | Cross-linking agent | Cumulant |
|---|---|---|---|
| particle | | (% by mass) relative | diameter |
| aqueous | Gelatin | to total amount of | (μm) of gelatin |
| solution | (% by mass) | gelatin | particles |
| A | 1 | 5 | 0.26 |
| B | 2 | 5 | 0.41 |
| C | 3 | 5 | 0.54 |
| D | 4 | 5 | — |
| E | 5 | 5 | — |

From the results of Table 1, it is seen that a lower gelatin concentration enabled gelatin particles with a smaller cumulant diameter, and that a higher gelatin concentration was more apt to generate coarse particles. It is also seen that a preferable gelatin concentration was 1% by mass or greater but 2% by mass or less in terms of securing yields, because when the gelatin concentration was 4% by mass or greater, the gelatin component did not become particles but became a block-state.

### Bioaffinity Particle Aqueous Solution Production Example 6

### <Production of Gelatin Particle Aqueous Solution F>

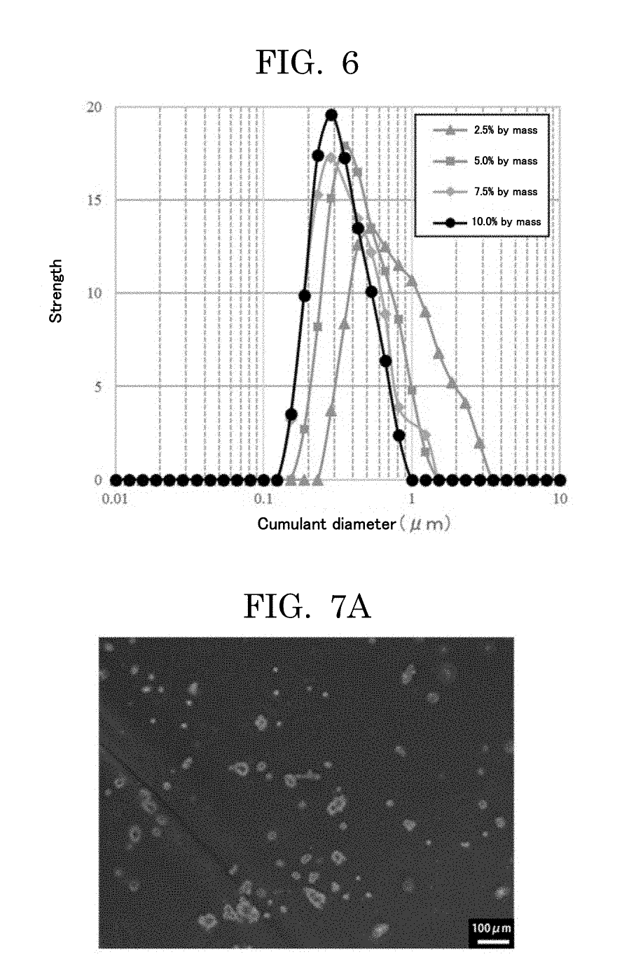

A gelatin particle aqueous solution F was obtained in the same manner as in Bioaffinity particle aqueous solution production example 2, except that unlike in Bioaffinity particle aqueous solution production example 2, the amount of the glutaraldehyde aqueous solution was changed from 160 μL (with a cross-linking agent of 5% by mass of the total amount of gelatin) to 80 μL (with a cross-linking agent of 2.5% by mass of the total amount of gelatin). The cumulant diameter of the gelatin particles in the obtained 2% by mass gelatin particle aqueous solution F is presented in Table 2 below. The particle size distribution of the gelatin particles is presented in FIG. 6.

### Bioaffinity Particle Aqueous Solution Production Example 7

### <Production of Gelatin Particle Aqueous Solution G>

A gelatin particle aqueous solution G was obtained in the same manner as in Bioaffinity particle aqueous solution production example 2, except that unlike in Bioaffinity particle aqueous solution production example 2, the amount of the glutaraldehyde aqueous solution was changed from 1604 (with a cross-linking agent of 5% by mass of the total amount of gelatin) to 240 μL (with a cross-linking agent of 7.5% by mass of the total amount of gelatin). The cumulant diameter of the gelatin particles in the obtained 2% by mass gelatin particle aqueous solution G is presented in Table 2 below. The particle size distribution of the gelatin particles is presented in FIG. 6.

### (Bioaffinity Particle Aqueous Solution Production Example 8)

### <Production of Gelatin Particle Aqueous Solution H>

A gelatin particle aqueous solution H was obtained in the same manner as in Bioaffinity particle aqueous solution production example 2, except that unlike in Bioaffinity particle aqueous solution production example 2, the amount of the glutaraldehyde aqueous solution was changed from 160 μL (with a cross-linking agent of 5% by mass of the total amount of gelatin) to 320 μL (with a cross-linking agent of 10.0% by mass of the total amount of gelatin). The cumulant diameter of the gelatin particles in the obtained 2% by mass gelatin particle aqueous solution H is presented in Table 2 below. The particle size distribution of the gelatin particles is presented in FIG. 6.

TABLE 2

| Gelatin | | Cross-linking agent | Cumulant |
|---|---|---|---|
| particle | | (% by mass) relative | diameter |
| aqueous | Gelatin | to total amount of | (μm) of gelatin |
| solution | (% by mass) | gelatin | particles |
| F | 2 | 2.5 | 0.69 |
| B | 2 | 5.0 | 0.41 |
| G | 2 | 7.5 | 0.33 |
| H | 2 | 10.0 | 0.32 |

From the results of Table 2, it is seen that a higher cross-linking agent concentration made the cumulant diameter of the gelatin particles smaller. This is estimated to be because the swelling rate of the gelatin particles varied depending on the cross-linking agent concentration, which led to differences in the cumulant diameter of the gelatin particles.

From the results of FIG. 6, it is seen that a higher cross-linking agent concentration made the particle size distribution narrower. This is estimated to be because a higher cross-linking agent concentration made the gelatin particles less likely to swell non-uniformly.

### Example 1

### <Production of Three-Dimensional Culture Structure>

Sodium alginate (product name: SKAT-ONE, available from Kimica Corporation) (0.02 g) was dissolved in water (10 mL), to prepare a 2% by mass sodium alginate aqueous solution (10 mL). To the resultant, the gelatin particle aqueous solution F (with a gelatin concentration of 2% by mass and a cross-linking agent concentration of 2.5% by mass) (10 mL) was added (at a volume ratio (2% by mass sodium alginate aqueous solution: gelatin particle aqueous solution F) of 1:1). The resultant was added into 6-well plate (product name COSTAR (registered trademark) cell culture plates, available from Corning Incorporated) on which BEMCOT (product name: BEMCOT M-1, available from Asahi Kasei Corporation) was laid, with a pipette by 3 mL/pipetting, and dried overnight at 60 degrees C. After drying, a 100 mmol/L (mM) calcium chloride aqueous solution (3 mL) was added into the 6-well plate, which was then left to stand still for 30 minutes or longer to form calcium alginate (cell support material), to obtain a three-dimensional culture structure containing gelatin particles (bioaffinity particles).

The obtained three-dimensional culture structure was washed with ion-exchanged water 3 times, and then washed with a 70% by mass ethanol aqueous solution 3 times or more.

In order to stabilize the thin film produced from the reaction between sodium alginate and calcium chloride, the thin film was immersed in a Dulbecco's modified Eagle's medium (D-MEM: available from Wako Pure Chemical Industries, Ltd.) for 72 hours under conditions of 37 degrees C. and 5% by volume CO₂. The medium was replaced every 24 hours.

### —Preparation of Cell Layer Forming Material—

Normal human dermal fibroblasts (NHDF, available from Lonza Japan Ltd.) subcultured 10 or more but 20 or less times were cultured in D-MEM (available from Wako Pure Chemical Industries, Ltd.) on a polystyrene dish for 72 hours under conditions of 37 degrees C. and 5% by volume CO₂. Subsequently, the cells were subjected to a trypsin treatment at 37 degrees C. for 5 minutes with a PBS containing a 0.05% by mass trypsin solution (Trypsin, from Porcine Pancreas, available from Wako Pure Chemical Industries, Ltd.) and 0.02% by mass ethylenediamine tetraacetic acid (EDTA, available from Wako Pure Chemical Industries, Ltd.) but free of calcium and magnesium, to strip the cells from the polystyrene dish. After the trypsin treatment, D-MEM containing 10% by mass FBS was added into the dish to terminate the enzyme reaction, to obtain a cell-containing solution. The obtained cell-containing solution (234 g) was subjected to a centrifugation treatment at 5 degrees C. for 5 minutes, and after the supernatant was removed, was suspended in D-MEM containing 10% by mass FBS, to obtain a cell layer forming material.

### —Cell Culture—

The cell layer forming material was applied on the three-dimensional culture structure that had been immersed in the D-MEM medium for 72 hours, in a manner that the number of cells applied was 4,000 cells/cm². Subsequently, the cell layer forming material was cultured for 48 hours under conditions of 37 degrees C. and 5% by volume CO₂. The medium was replaced once 24 hours after the start of the culture.

### (Adhesion and Stretching of Cells)

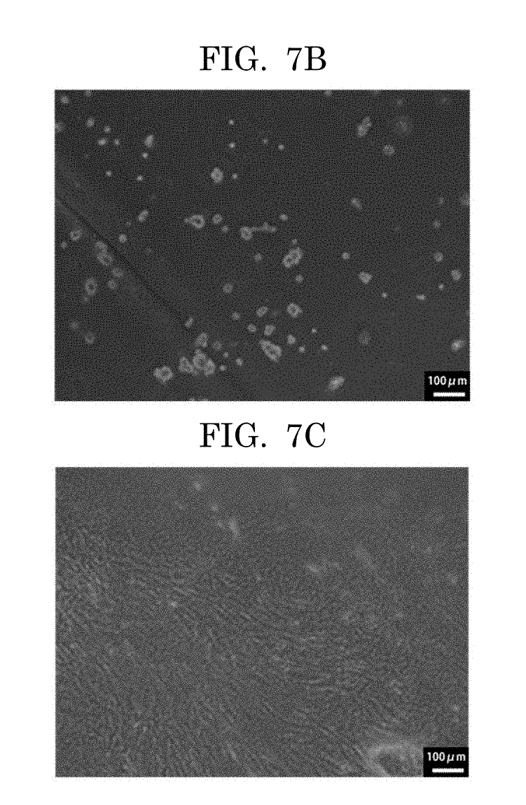

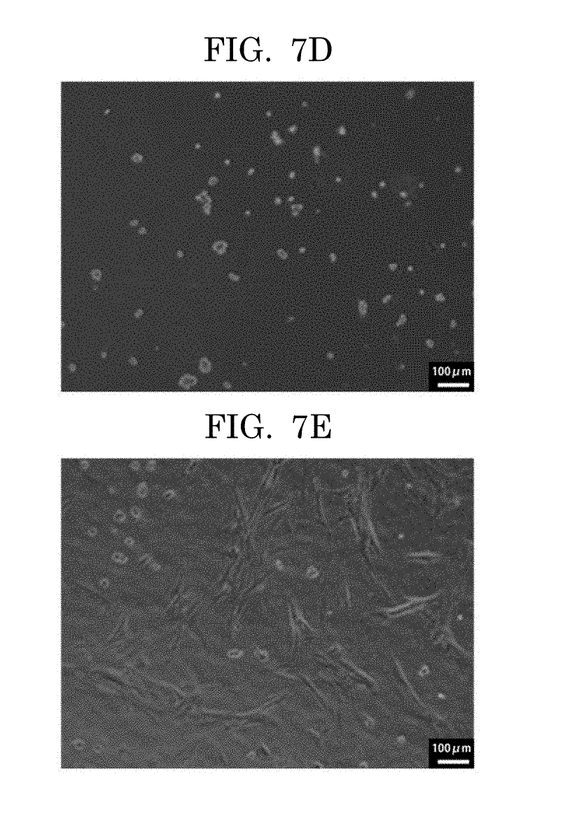

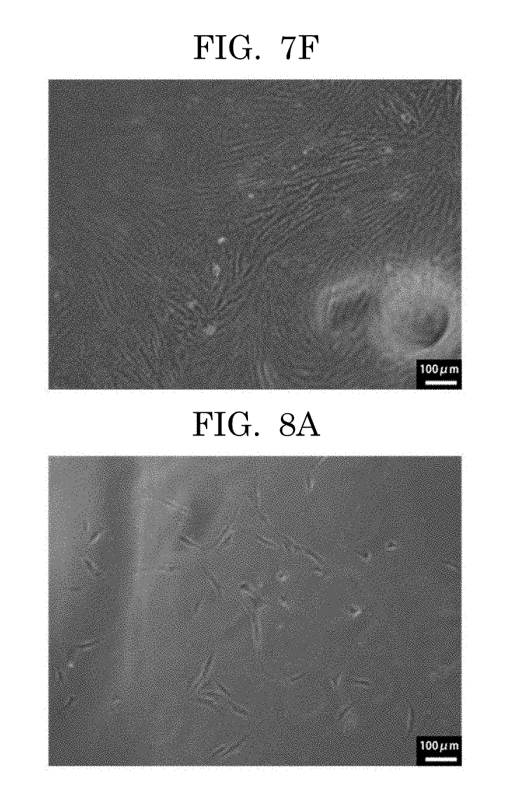

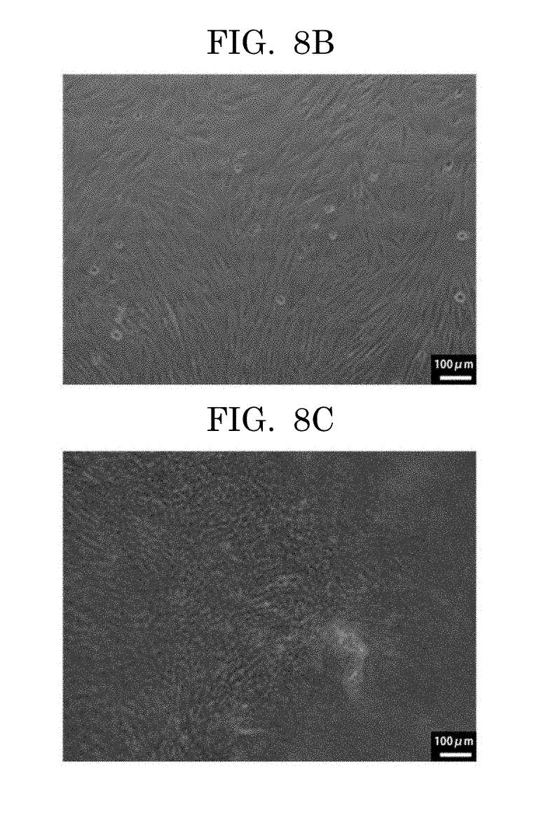

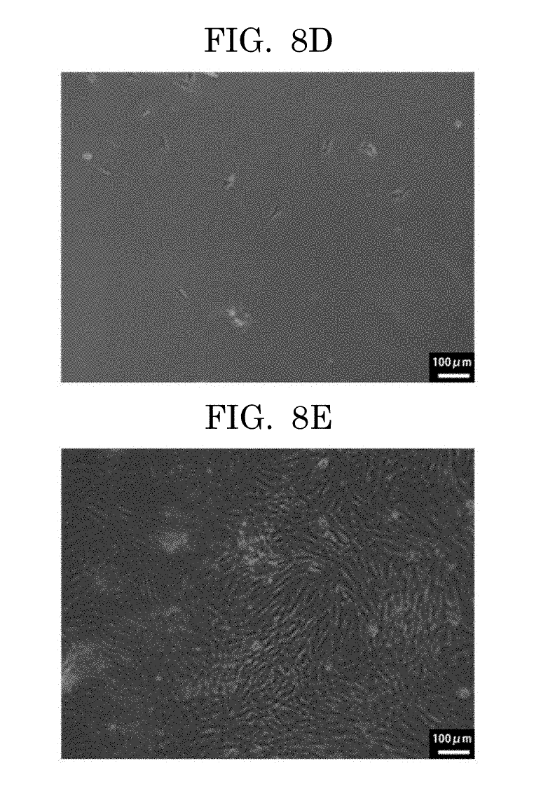

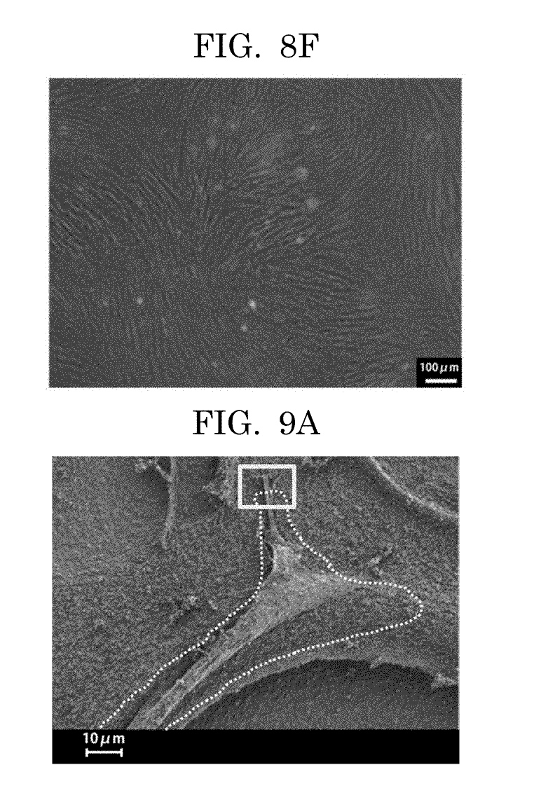

During the cell culture, adhesion and stretching of the cells were observed with a phase-contrast microscope 24 hours and 48 hours after the start of the culture. The results are presented in FIG. 7B (24 hours after) and FIG. 8B (48 hours after).

Using IMAGE J (software), the number of cells was counted from fluorescence images captured with the phase-contrast microscope. The number of cells was calculated based on the average of 6 images. The stretching rate was also obtained by counting the number of cells that had stretched. The result of the stretching rate is presented in Table 3 below.

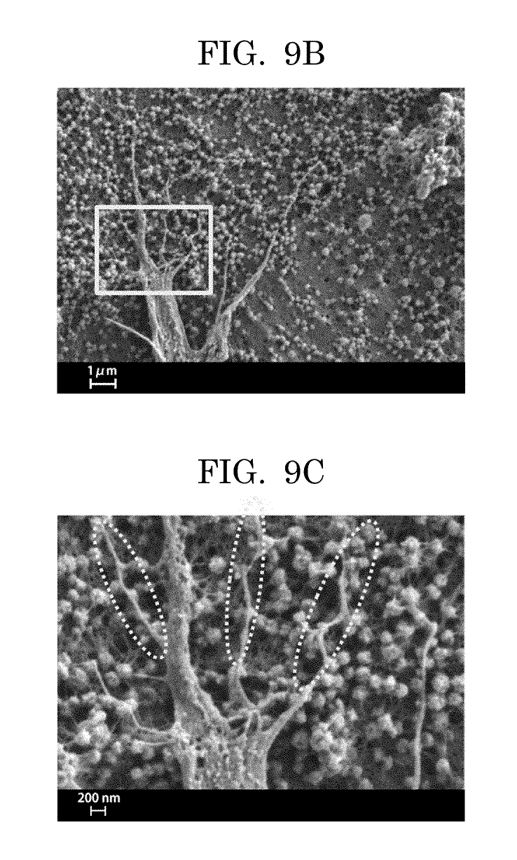

The observation results of the fluorescence images are presented in FIG. 9A to FIG. 9C. FIG. 9A is a photograph presenting a state of the cells after culture for 48 hours in Example 1. In FIG. 9A, the dashed line indicates a stretched cell. FIG. 9B presents a partially enlarged photograph of the solid line portion in FIG. 9A. FIG. 9C presents a partially enlarged photograph of the solid line portion in FIG. 9B. From FIG. 9C, it can be seen that pseudopods of a cell stretched in a manner of creeping regions where gelatin particles densely concentrated.

### Example 2

Adhesion and stretching of cells were observed in the same manner as in Example 1, except that unlike in Example 1, the gelatin particle aqueous solution F (with a gelatin concentration of 2% by mass and a cross-linking agent concentration of 2.5% by mass) (10 mL) was changed to the gelatin particle aqueous solution G (with a gelatin concentration of 2% by mass and a cross-linking agent concentration of 7.5% by mass) (10 mL). The results are presented in Table 3 below, FIG. 7C (24 hours after), and FIG. 8C (48 hours after).

### Comparative Example 1

Adhesion and stretching of cells were observed in the same manner as in Example 1, except that unlike in Example 1, the gelatin particle aqueous solution F (with a gelatin concentration of 2% by mass and a cross-linking agent concentration of 2.5% by mass) (10 mL) was not used. The results are presented in Table 3 below, FIG. 7A (24 hours after), and FIG. 8A (48 hours after). Further, in the same manner as in Example 1, the cell stretching rate was obtained. The result is presented in Table 3 below.

### Example 3

Adhesion and stretching of cells were observed in the same manner as in Example 1, except that unlike in Example 1, the 100 mmol/L calcium chloride aqueous solution (3 mL) was changed to a 500 mmol/L (mM) calcium chloride aqueous solution (3 mL). The results are presented in Table 3 below, FIG. 7E (24 hours after), and FIG. 8E (48 hours after). Further, in the same manner as in Example 1, the cell stretching rate was obtained. The result is presented in Table 3 below.

### Example 4

Adhesion and stretching of cells were observed in the same manner as in Example 2, except that unlike in Example 2, the 100 mmol/L calcium chloride aqueous solution (3 mL) was changed to a 500 mmol/L (mM) calcium chloride aqueous solution (3 mL). The results are presented in Table 3 below, FIG. 7F (24 hours after), and FIG. 8F (48 hours after). Further, in the same manner as in Example 1, the cell stretching rate was obtained. The result is presented in Table 3 below.

### Comparative Example 2

Adhesion and stretching of cells were observed in the same manner as in Example 3, except that unlike in Example 3, the gelatin particle aqueous solution F (with a gelatin concentration of 2% by mass and a cross-linking agent concentration of 2.5% by mass) (10 mL) was not used. The results are presented in Table 3 below, FIG. 7D (24 hours after), and FIG. 8D (48 hours after). Further, in the same manner as in Example 1, the cell stretching rate was obtained. The result is presented in Table 3 below.

TABLE 3

| | | | | | | Presence or | | | | |
|---|---|---|---|---|---|---|---|---|---|---|
| | | Bioaffinity particles | | Cell support | Surface | absence of | | | | |
| | | (gelatin particles) | | material pre- | area | protrusion | | | | |
| | Cell support | | Cross- | cursor gelating | occupation | of bioaffinity | Evaluation result | | | |
| | material pre- | | linking agent | polymer (calcium | rate (%) of | particles to | Cell adhesion/ | | Cell stretching | |
| | cursor (sodium | | (% by mass) to | chloride) con- | exposed | 3D culture | stretching | | rate (%) | |
| | alginate) | Gelatin | total amount | centration | bioaffinity | structure | 24 hours | 48 hours | 24 hours | 48 hours |
| | (% by mass) | (% by mass) | of gelatin | (mmol/L) | particles | surface | after | after | after | after |
| Comp. | 2 | — | — | 100 | — | — | FIG. 7A | FIG. 8A | 0 | 0 |
| Ex. 1 | | | | | | | | | | |
| Ex. 1 | 2 | 2 | 2.5 | 100 | 58 | Present | FIG. 7B | FIG. 8B | 90 | 95 |
| Ex. 2 | 2 | 2 | 7.5 | 100 | 50 | Present | FIG. 7C | FIG. 8C | 85 | 95 |
| Comp. | 2 | — | — | 500 | — | — | FIG. 7D | FIG. 8D | 0 | 0 |
| Ex. 2 | | | | | | | | | | |
| Ex. 3 | 2 | 2 | 2.5 | 500 | 18 | Present | FIG. 7E | FIG. 8E | 50 | 80 |
| Ex. 4 | 2 | 2 | 7.5 | 500 | 50 | Present | FIG. 7F | FIG. 8F | 50 | 95 |

From the results of FIG. 7A to FIG. 7F and FIG. 8A to FIG. 8F, it can be seen that the three-dimensional culture structures containing gelatin particles, which were bioaffinity particles, and calcium alginate, which was a cell support material were suitable for use in cell culture, because cells cultured on the three-dimensional culture structures were promoted to adhere and stretch.

The three-dimensional culture structures of Examples 1 to 4 were to measured by gel permeation chromatography (GPC). As a result, it was confirmed that gelatin particles and calcium alginate were contained.

### Example 5

### <Production of Three-Dimensional Culture Structure>

Sodium alginate (product name: SKAT-ONE, available from Kimica Corporation) (0.01 g) was dissolved in water (10 mL), to prepare a 1% by mass sodium alginate aqueous solution (10 mL). To the resultant, a gelatin particle aqueous solution F diluted liquid (10 mL) obtained by diluting the gelatin particle aqueous solution F (with a gelatin concentration of 2% by mass and a cross-linking agent concentration of 2.5% by mass) to a gelatin concentration of 1% was added (at a volume ratio (1% by mass sodium alginate aqueous solution:gelatin particle aqueous solution F 1%) of 1:1), to obtain a mixture liquid A. Next, with an industrial inkjet head GEN4 (available from Ricoh Industry Company, Ltd.), the obtained mixture liquid A was printed on 6-well plate (product name COSTAR (registered trademark) cell culture plates, available from Corning Incorporated) in a manner that a layer having a dimension of 8 mm on each side and an average thickness of 30 μm was formed, and then 100 mM calcium chloride was overprinted to have an average thickness of 10 μm on the mixture liquid A, to form a structure. Subsequently, the obtained structure was washed once with a serum-free Dulbecco's modified Eagle's medium (available from Life Technology Corporation), to obtain a three-dimensional culture structure.

### —Cell Staining—

A cryopreserved green fluorescent dye (product name: CELL TRACKER GREEN, available from Life Technology Corporation) was defrosted to room temperature, and dissolved at a concentration of 10 mM in dimethylsulfoxide (hereinafter may also be referred to as “DMSO”). The resultant was mixed with a serum-free Dulbecco's modified Eagle's medium (available from Life Technology Corporation), to prepare a green fluorescent dye-containing serum-free medium having a concentration of 10 μM. Next, the prepared green fluorescent dye-containing serum-free medium was added to the cell layer forming material prepared in Example 1, in an amount of 5 mL per dish, to stain the cell layer forming material in an incubator for 30 minutes, to obtain a cell layer forming material containing cells stained with the green fluorescent dye.

### —Cell Culture—

The cell layer forming material containing cells stained with the green fluorescent dye was applied on the obtained three-dimensional culture structure in a manner that the number of cells applied was 4,000 cells/cm². Subsequently, the cells were cultured for 48 hours under conditions of 37 degrees C. and 5% by volume CO₂. The medium (serum-free Dulbecco's modified. Eagles medium, available from Life Technology Corporation) was replaced once 24 hours after the start of the culture.

### (Adhesion and Stretching of Cells)

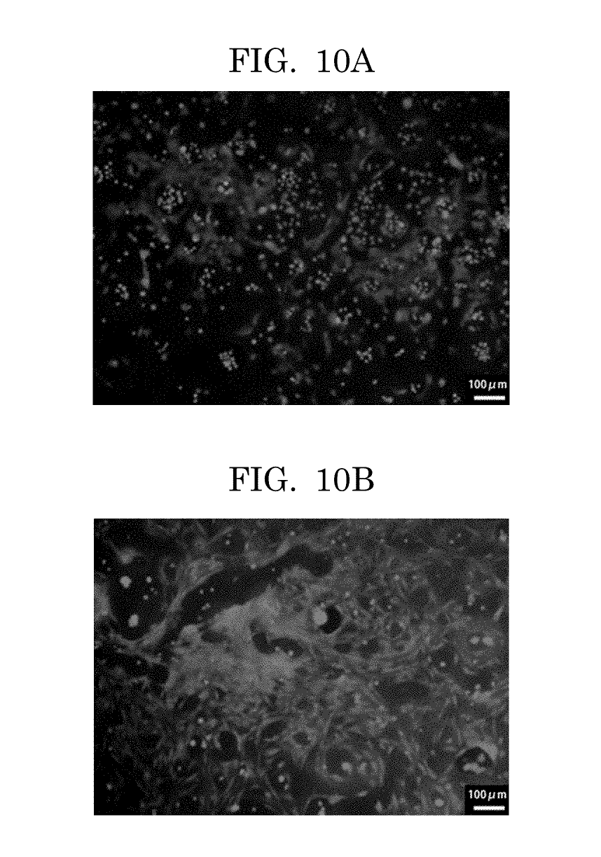

During the cell culture, adhesion and stretching of the cells were observed with a phase-contrast microscope 4 hours and 24 hours after the start of the culture. The results are presented in FIG. 10A (4 hours after) and FIG. 10B (24 hours after). The stretching rate 4 hours after was 50% and the stretching rate 24 hours after was 90%. The results are presented in Table 4 below.

### Example 6

A three-dimensional culture structure was obtained in the same manner as in Example 5, except that unlike in Example 5, the mixture liquid A was changed to a mixture liquid B obtained by dissolving sodium alginate (product name: SKAT-ONE, available from Kimica Corporation) (0.005 g) in water (10 mL) to prepare a 0.5% by mass sodium alginate aqueous solution (10 mL), diluting the gelatin particle aqueous solution B (with a gelatin concentration of 2% by mass and a cross-linking agent concentration of 5% by mass) to a gelatin concentration of 0.5% by mass to obtain a gelatin particle aqueous solution B diluted liquid, and adding the gelatin particle aqueous solution B diluted liquid (10 mL) to the 0.5% by mass sodium alginate aqueous solution (10 mL) (at a volume ratio (0.5% by mass sodium alginate aqueous solution:0.5% by mass gelatin particle aqueous solution B) of 1:1).

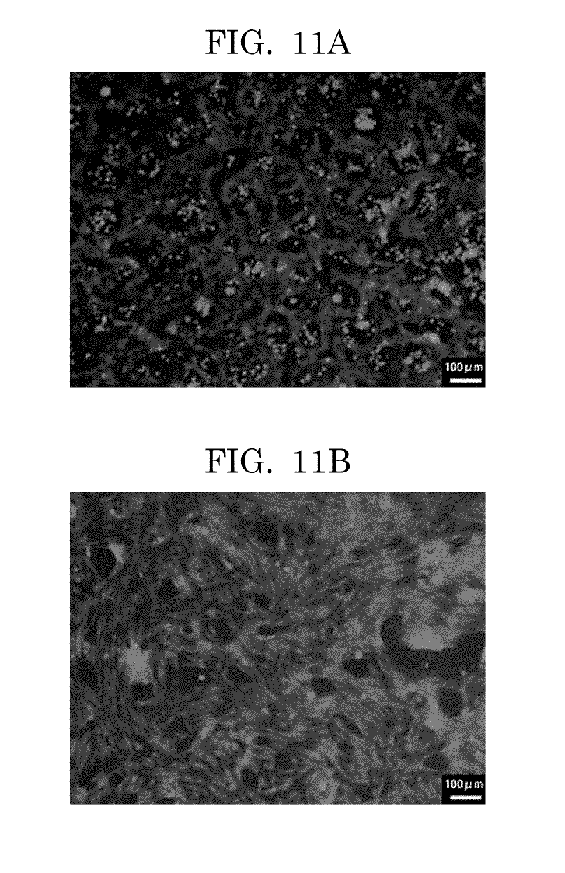

Next, cells were cultured in the same manner as in Example 5, and adhesion and stretching of the cells were observed in the same manner as in Example 5. The results are presented in FIG. 11A (4 hours after) and FIG. 11B (24 hours after). The stretching rate 4 hours after was 60% and the stretching rate 24 hours after was 98%. The results are presented in Table 4 below.

### Comparative Example 3

A three-dimensional culture structure was obtained in the same manner as in Example 6, except that unlike in Example 6, the gelatin particle aqueous solution B was not added.

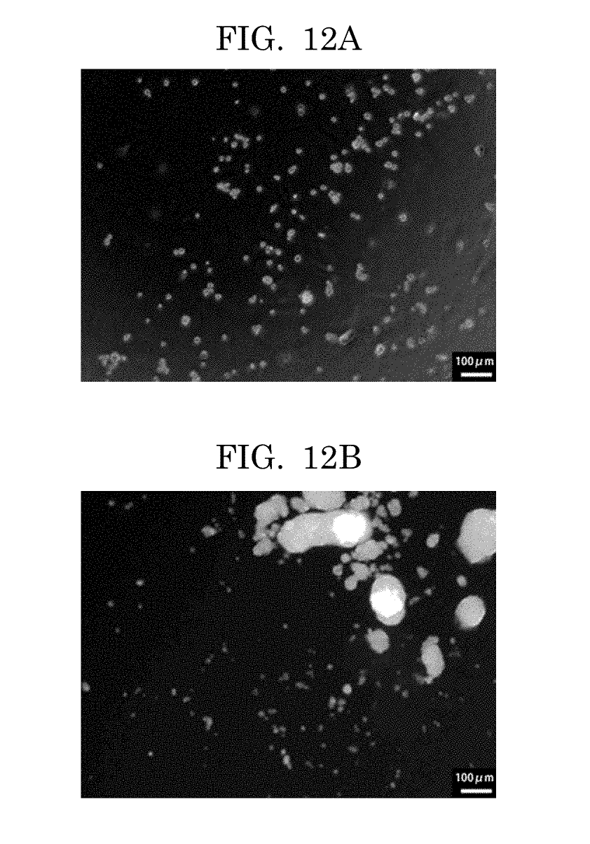

Next, cells were cultured in the same manner as in Example 6, and adhesion and stretching of the cells were observed in the same manner as in Example 6. The results are presented in FIG. 12A (4 hours after) and FIG. 12B (24 hours after). The stretching rate 4 hours after was 0% and the stretching rate 24 hours after was 0%. The results are presented in Table 4 below.

### Comparative Example 4

An attempt was made to produce a three-dimensional culture structure in the same manner as in Example 5, except that unlike in Example 5, the gelatin particle aqueous solution F was changed to an aqueous solution of non-particulate gelatin (with a gelatin concentration of 1% by mass). However, the liquid was not able to be discharged stably. Hence, the alginic acid-gelatin mixture liquid was manually applied on a well. Next, in the same manner as in Example 5, 100 mM calcium chloride was overprinted to have an average thickness of 10 μm on the mixture liquid, to obtain a three-dimensional culture structure.

Next, cells were cultured in the same manner as in Example 5. As a result, the three-dimensional culture structure collapsed.

TABLE 4

| | | | | | Presence or | | | | |
|---|---|---|---|---|---|---|---|---|---|
| | | | | | absence of | | | | |
| | | Concentration | Concentration | Surface area | protrusion | | | | |
| | | (% by mass) | (% by mass) | occupation | of bioaffinity | Evaluation result | | | |
| | | of sodium | of gelatin | rate (%) of | particles to | Cell adhesion/ | | Cell stretching | |
| | Kind of | alginate in | particles in | exposed | 3D culture | stretching | | rate (%) | |
| | gelatin | 3D culture | 3D culture | bioaffinity | structure | 4 hours | 24 hours | 4 hours | 24 hours |
| | particles | structure | structure | particles | surface | after | after | after | after |
| Ex. 5 | F | 0.5 | 0.5 | 58 | Present | FIG. 10A | FIG. 10B | 50 | 90 |
| Ex. 6 | B | 0.25 | 0.25 | 58 | Present | FIG. 11A | FIG. 11B | 60 | 98 |
| Comp. | — | 0.25 | — | — | — | FIG. 12A | FIG. 12B | 0 | 0 |
| Ex. 3 | | | | | | | | | |

| | | | | | | | | |
|---|---|---|---|---|---|---|---|---|
| Comp. | — | 0.5 | — | — | — | — | — | Unmeasurable |
| Ex. 4 | | | | | | | | |

### Example 7

Sodium alginate (product name: SKAT-ONE, available from Kimica Corporation) (0.02 g) was dissolved in water (10 mL), to prepare a 2% by mass sodium alginate aqueous solution (10 mL). To the resultant, a polylactic acid particle solution (with a polylactic acid concentration of 2% by mass, with PLA particles of 250 nm, available from Corefront Corporation) (10 mL) was added (at a volume ratio (1% by mass sodium alginate aqueous solution:1% by mass polylactic acid particle solution) of 1:1), to obtain a mixture liquid C.

Next, a three-dimensional culture structure was obtained in the same manner as in Example 5, except that unlike in Example 5, the mixture liquid A was changed to the mixture liquid C.

Next, cells were cultured in the same manner as in Example 5, and the stretching rate was measured in the same manner as in Example 5. The stretching rate 4 hours after was 10% and the stretching rate 24 hours after was 50%. The results are presented in Table 5 below.

### Example 8

A three-dimensional culture structure was obtained in the same manner as in Example 7, except that unlike in Example 7, the polylactic acid particle solution was changed to a polystyrene particle solution (with a polystyrene concentration of 2% by mass, MICROMER-REDF available from Corefront Corporation), and the amount of addition was at a volume ratio (1% by mass sodium alginate aqueous solution:1% by mass polystyrene particle solution) of 1:1.

Next, cells were cultured in the same manner as in Example 5, and the stretching rate was measured in the same manner as in Example 7. The stretching rate 4 hours after was 10% and the stretching rate 24 hours after was 50%. The results are presented in Table 5 below.

### Example 9

A three-dimensional culture structure was obtained in the same manner as in Example 7, except that unlike in Example 7, the polylactic acid particle solution was changed to a silica particle solution (with a silica concentration of 4% by mass, SICASTAR-REDF available from Corefront Corporation), and the amount of addition was at a volume ratio (1% by mass sodium alginate aqueous solution:2% by mass silica particle solution) of 1:1.

Next, cells were cultured in the same manner as in Example 5, and the stretching rate was measured in the same manner as in Example 7. The stretching rate 4 hours after was 10% and the stretching rate 24 hours after was 40%. The results are presented in Table 5 below.

### Example 10

A three-dimensional culture structure was obtained in the same manner as in Example 7, except that unlike in Example 7, the polylactic acid particle solution was changed to a gelatin particle aqueous solution F diluted liquid obtained by diluting the gelatin particle aqueous solution F (with a gelatin concentration of 2% by mass and a cross-linking agent concentration of 2.5% by mass) to a gelatin concentration of 1% by mass, and the amount of addition was at a volume ratio (1% by mass sodium alginate aqueous solution:1% by mass gelatin particle aqueous solution F diluted liquid) of 1:1.

Next, cells were cultured in the same manner as in Example 5, and the stretching rate was measured in the same manner as in Example 7. The stretching rate 4 hours after was 50% and the stretching rate 24 hours after was 90%. The results are presented in Table 5 below.

TABLE 5

| | | Concentration | Concentration | Surface area | Presence or absence | | |
|---|---|---|---|---|---|---|---|
| | | (% by mass) | (% by mass) | occupation | of protrusion | Evaluation result | |
| | | of sodium | of bioaffinity | rate (%) | of bioaffinity | Cell stretching | |
| | Kind of | alginate in | particles in | of exposed | particles to | rate (%) | |
| | bioaffinity | 3D culture | 3D culture | bioaffinity | 3D culture | 4 hours | 24 hours |
| | particles | structure | structure | particles | structure surface | after | after |
| Ex. 7 | Polylactic acid | 1.0 | 1.0 | 20 | Present | 10 | 50 |
| | particles | | | | | | |
| Ex. 8 | Polystyrene | 1.0 | 1.0 | 20 | Present | 10 | 50 |
| | particles | | | | | | |
| Ex. 9 | Silica | 1.0 | 2.0 | 20 | Present | 10 | 40 |
| | particles | | | | | | |
| Ex. 10 | Gelatin particle | 1.0 | 1.0 | 58 | Present | 50 | 90 |
| | aqueous solution F | | | | | | |

Aspects of the present invention are as follows, for example.

<1> A three-dimensional culture structure including:
cells;
a cell support material configured to support the cells; and bioaffinity particles.
<2> The three-dimensional culture structure according to <1>, wherein the bioaffinity particles are exposed from at least part of a surface of the cell support material.
<3> The three-dimensional culture structure according to <2>, wherein the bioaffinity particles are protruded from the cell support material.
<4> The three-dimensional culture structure according to <2> or <3>, wherein a surface area occupation rate at which the bioaffinity particles are exposed is 20% or greater of an entire surface of the three-dimensional culture structure.
<5> The three-dimensional culture structure according to <1>, wherein the bioaffinity particles are dispersed in the cell support material.
<6> The three-dimensional culture structure according to any one of <1> to <5>,
wherein the bioaffinity particles have a point of adhesion to adhere with the cells.
<7> The three-dimensional culture structure according to any one of <1> to <6>,
wherein a cumulant diameter of the bioaffinity particles is 0.1 μm or greater but 1.0 μm or less.
<8> The three-dimensional culture structure according to any one of <1> to <7>,
wherein the bioaffinity particles include gelatin particles.
<9> The three-dimensional culture structure according to any one of <1> to <8>,
wherein the cell support material includes a polysaccharide.
<10> The three-dimensional culture structure according to <9>,
wherein the polysaccharide includes calcium alginate.
<11> The three-dimensional culture structure according to any one of <1> to <10>,
wherein a content of the bioaffinity particles is 0.5% by mass or greater but 2% by mass or less.
<12> The three-dimensional culture structure according to any one of <1> to <11>,
wherein the cells are adherent cells.
<13> A method for producing a three-dimensional culture structure, the method including:
a layer forming step of forming a cell support material precursor aqueous solution layer on a base material, the cell support material precursor aqueous solution layer being formed of a cell support material precursor aqueous solution containing bioaffinity particles and a cell support material precursor;
a cell support material precursor gelating aqueous solution applying step of applying a cell support material precursor gelating aqueous solution on the cell support material precursor aqueous solution layer, the cell support material precursor gelating aqueous solution being configured to gelate the cell support material precursor upon contact with the cell support material precursor aqueous solution; and
a cell layer forming material applying step of applying a cell layer forming material containing cells,
wherein the method produces the three-dimensional culture structure according to any one of <1> to <12>.
<14> The method for producing a three-dimensional culture structure according to <13>,
wherein forming the cell support material precursor aqueous solution layer on the base material in the layer forming step is performed by discharging the cell support material precursor aqueous solution onto the base material, and
wherein applying the cell support material precursor gelating aqueous solution on the cell support material precursor aqueous solution layer in the cell support material precursor gelating aqueous solution applying step is performed by discharging the cell support material precursor gelating aqueous solution onto the cell support material precursor aqueous solution layer.
<15> The method for producing a three-dimensional culture structure according to <14>,
wherein discharging the cell support material precursor aqueous solution onto the base material and discharging the cell support material precursor gelating aqueous solution onto the cell support material precursor aqueous solution layer are performed according to an inkjet method.
<16> The method for producing a three-dimensional culture structure according to any one of <13> to <15>,
wherein the cell support material precursor gelating aqueous solution is at least one selected from the group consisting of a calcium chloride aqueous solution, a chitosan aqueous solution, and a chitin aqueous solution.
<17> The method for producing a three-dimensional culture structure according to any one of <13> to <16>,
wherein the cell support material precursor aqueous solution is at least one selected from the group consisting of a sodium alginate aqueous solution, a potassium alginate aqueous solution, and an ammonium alginate aqueous solution.
<18> The method for producing a three-dimensional culture structure according to any one of <13> to <17>, wherein a viscosity of the cell support material precursor aqueous solution is 20 mPa·s or less.
<19> The method for producing a three-dimensional culture structure according to any one of <13> to <18>,
wherein the cells are adherent cells.
<20> The method for producing a three-dimensional culture structure according to <19>,
wherein the adherent cells are fibroblasts.

The three-dimensional culture structure according to any one of <1> to <12> and the method for producing a three-dimensional culture structure according to any one of <13> to <20> can solve the various problems in the related art and can achieve the object of the present invention.

What is claimed is:

1. A three-dimensional culture structure comprising:
cells;
a cell support material configured to support the cells; and
bioaffinity particles.

2. The three-dimensional culture structure according to claim 1, wherein the bioaffinity particles are exposed from at least part of a surface of the cell support material.

3. The three-dimensional culture structure according to claim 2, wherein the bioaffinity particles are protruded from the cell support material.

4. The three-dimensional culture structure according to claim 2, wherein a surface area occupation rate at which the bioaffinity particles are exposed is 20% or greater of an entire surface of the three-dimensional culture structure.

5. The three-dimensional culture structure according to claim 1, wherein the bioaffinity particles are dispersed in the cell support material.

6. The three-dimensional culture structure according to claim 1, wherein the bioaffinity particles have a point of adhesion to adhere with the cells.

7. The three-dimensional culture structure according to claim 1, wherein a cumulant diameter of the bioaffinity particles is 0.1 μm or greater but 1.0 μm or less.

8. The three-dimensional culture structure according to claim 1, wherein the bioaffinity particles comprise gelatin particles.

9. The three-dimensional culture structure according to claim 1, wherein the cell support material comprises a polysaccharide.

10. The three-dimensional culture structure according to claim 9, wherein the polysaccharide comprises calcium alginate.

11. The three-dimensional culture structure according to claim 1, wherein a content of the bioaffinity particles is 0.5% by mass or greater but 2% by mass or less.

12. The three-dimensional culture structure according to claim 1, wherein the cells comprise adherent cells.

13. A method for producing a three-dimensional culture structure, the method comprising:
forming a cell support material precursor aqueous solution layer on a base material, the cell support material precursor aqueous solution layer being formed of a cell support material precursor aqueous solution that comprises bioaffinity particles and a cell support material precursor;
applying a cell support material precursor gelating aqueous solution on the cell support material precursor aqueous solution layer, the cell support material precursor gelating aqueous solution being configured to gelate the cell support material precursor upon contact with the cell support material precursor aqueous solution; and
applying a cell layer forming material that comprises cells, wherein the method produces a three-dimensional culture structure that comprises:
cells;
a cell support material configured to support the cells; and
bioaffinity particles.

14. The method for producing a three-dimensional culture structure according to claim 13,
wherein forming the cell support material precursor aqueous solution layer on the base material is performed by discharging the cell support material precursor aqueous solution onto the base material, and
wherein applying the cell support material precursor gelating aqueous solution on the cell support material precursor aqueous solution layer is performed by discharging the cell support material precursor gelating aqueous solution onto the cell support material precursor aqueous solution layer.

15. The method for producing a three-dimensional culture structure according to claim 14,
wherein discharging the cell support material precursor aqueous solution onto the base material and discharging the cell support material precursor gelating aqueous solution onto the cell support material precursor aqueous solution layer are performed according to an inkjet method.

16. The method for producing a three-dimensional culture structure according to claim 13,
wherein the cell support material precursor gelating aqueous solution comprises at least one selected from the group consisting of a calcium chloride aqueous solution, a chitosan aqueous solution, and a chitin aqueous solution.

17. The method for producing a three-dimensional culture structure according to claim 13,
wherein the cell support material precursor aqueous solution comprises at least one selected from the group consisting of a sodium alginate aqueous solution, a potassium alginate aqueous solution, and an ammonium alginate aqueous solution.

18. The method for producing a three-dimensional culture structure according to claim 13,
wherein a viscosity of the cell support material precursor aqueous solution is 20 mPa·s or less.

19. The method for producing a three-dimensional culture structure according to claim 13,
wherein the cells comprise adherent cells.

20. The method for producing a three-dimensional culture structure according to claim 19,
wherein the adherent cells comprise fibroblasts.
